SEQUENCE LISTING (5 sequences)

SEQ ID NO: 1 - DNA, 21 bp, Artificial Sequence
  Synthetic oligonucleotide
ggcagcctttgggatcccaac

SEQ ID NO: 2 - DNA, 21 bp, Artificial Sequence
  Synthetic oligonucleotide
ctactcttctcaccagcgcgc

SEQ ID NO: 3 - DNA, 22 bp, Artificial Sequence
  Synthetic oligonucleotide
gtgcccacagagtgtcttctgt

SEQ ID NO: 4 - DNA, 23 bp, Artificial Sequence
  Synthetic oligonucleotide
gcggtctggcagtaaaaactatc

SEQ ID NO: 5 - DNA, 23 bp, Artificial Sequence
  Synthetic oligonucleotide
gtgaaacagcattgctgtcactt

(19) United States
(12) Patent Application Publication
(10) Pub. No.: US 2017/0326356 A1
(43) Pub. Date: Nov. 16, 2017

(54) METHODS FOR TREATING PSYCHIATRIC DISORDERS

(71) Applicants: Jun R. HUH, Newton, MA (US); Bohyun Gloria CHOI, Newton, MA (US); Dan R. LITTMAN, New York, NY (US); Yeong Shin YIM, Cambridge, MA (US)

(72) Inventors: Jun R. HUH, Newton, MA (US); Bohyun Gloria CHOI, Newton, MA (US); Dan R. LITTMAN, New York, NY (US); Yeong Shin YIM, Cambridge, MA (US)

(21) Appl. No.: 15/416,238

(22) Filed: Jan. 26, 2017

Related U.S. Application Data
(60) Provisional application No. 62/287,104, filed on Jan. 26, 2016.

(51) Int. Cl. A61N 1/05; A61B 5/00; C12Q 1/68; A61N 1/36

(52) U.S. Cl. CPC A61N 1/0531; C12Q 1/6883; A61B 5/0006; A61N 1/36

(57) ABSTRACT

Methods for treating subjects afflicted with a psychiatric disorders [e.g., autism spectrum disorder (ASD), schizophrenia, and/or depression] are described herein. More particularly, the present invention relates to a method for treating a subject afflicted with or at risk for developing a psychiatric disorder (e.g., ASD, of which autism is a particular example) that calls for selecting a subject afflicted with or at risk for developing the psychiatric disorder based in part on previous in utero exposure to maternal immune activation (MIA) and treating the subject by administering pharmacological agents or implementing optogenetic tools or chemogenetic tools that correct dysregulated neuronal excitation/inhibition (E/I) ratios in cortical patches of the subject wherein the E/I ratio is dysregulated. A subject may also be selected for treatment using methods described herein based on the presence or detection of cortical patches having dysregulated neuronal E/I ratios.

### CROSS REFERENCE TO RELATED APPLICATIONS

This application claims priority under 35 USC §119(e) from U.S. Provisional Application Ser. No. 62/287,104, filed Jan. 26, 2016, which application is herein specifically incorporated by reference in its entirety.

### FIELD OF THE INVENTION

The present invention relates to treating subjects afflicted with a psychiatric disorder [e.g., autism spectrum disorder (ASD), schizophrenia, and/or depression]. More particularly, the present invention relates to a method for treating a subject afflicted with or at risk for developing a psychiatric disorder (e.g., ASD, of which autism is a particular example) that calls for selecting a subject afflicted with or at risk for developing the psychiatric disorder based in part on previous in utero exposure to maternal immune activation (MIA) and treating the subject (i.e., offspring that is no longer in utero) by administering pharmacological agents [such as, for example, γ-Aminobutyric acid (GABA)ergic receptor agonists] or implementing optogenetic tools or chemogenetic tools to correct dysregulated neuronal excitation/inhibition (E/I) ratios in cortical patches of the subject wherein the E/I ratio is dysregulated. A subject may also be selected for treatment using methods described herein based on the presence or detection of cortical patches having dysregulated neuronal E/I ratios.

### BACKGROUND OF THE INVENTION

Viral infection during pregnancy has been correlated with increased frequency of autism spectrum disorder (ASD) in offspring¹⁻⁶. This phenomenon has been modeled in mice prenatally subjected to maternal immune activation (MIA)⁷⁻¹⁰. We previously showed that the T helper 17 (Th17) cell/interleukin-17a (IL-17a) pathway is crucial for the induction of both cortical and behavioral abnormalities observed in MIA-affected offspring¹¹. It, however, remains unclear if and how cortical abnormalities serve as causative factors for the aberrant behavioral phenotypes. The mechanism whereby MIA contributes to the development of autism and the specific immune cell population(s) involved are, moreover, the focus of ongoing scientific research, as are novel therapeutic approaches for treating subjects afflicted with autism.

The citation of references herein shall not be construed as an admission that such is prior art to the present invention.

### SUMMARY OF THE INVENTION

Results presented herein show that cortical abnormalities are preferentially localized to a region encompassing the dysgranular zone of the primary somatosensory cortex (S1DZ) in adult MIA offspring (adults that were prenatally subjected to MIA) and that the presence and size of cortical patches tightly correlate with manifestation and severity of ASD-like behavioral phenotypes. More specifically, the present inventors demonstrate that the selective loss of parvalbumin (PV)-expressing interneurons and a concomitant increase in neural activity is causal to the emergence of MIA behavioral phenotypes. Indeed, activation of pyramidal neurons in the S1DZ was sufficient to induce ASD-like behaviors in wild-type (WT) animals, while reduction in neural activity in this cortical region rescued the behavioral abnormalities in the MIA offspring. The present inventors also identified the temporal association area (TeA) as a S1DZ downstream target involved in the selective modulation of sociability phenotypes, but not the expression of repetitive behaviors. Accordingly, results presented herein identify a cortical region primarily, if not exclusively, centered on the S1DZ as the major node of a neural network whose increased neural activity mediates ASD-like behavioral abnormalities observed in offspring exposed to maternal inflammation in utero.

In keeping with discoveries presented herein, a method for treating a subject afflicted with or at risk for developing a psychiatric disorder (e.g., ASD, of which autism is a particular example) that calls for selecting a subject afflicted with or at risk for developing the psychiatric disorder based at least in part on previous in utero exposure to MIA (prenatal exposure) and treating the subject (i.e., offspring that is no longer in utero) by administering pharmacological agents such as, for example, GABAergic receptor agonists or implementing optogenetic tools or chemogenetic tools to correct dysregulated neuronal E/I ratios in cortical patches of the subject wherein the E/I ratio is dysregulated. In a particular embodiment, the prenatal exposure occurs in the late first trimester or the second trimester of a pregnancy in a mammal and more particularly, in a human. In a further embodiment, the exposure to prenatal MIA is detected and/or confirmed by measuring elevated levels of IL-17a in the mother's serum during the pregnancy. In another embodiment, the patches of cortical malformation are located in the S1DZ. In another particular embodiment, the pharmacological agents and/or optogenetic tools are administered to the cortex of the subject to at least partially restore a normal E/I ratio (comparable to wildtype E/I ratio) in patches of cortical malformation wherein the E/I ratio is dysregulated in the absence of treatment. In a more particular embodiment thereof, the pharmacological agents and/or optogenetic tools are administered to and/or in the vicinity of the S1DZ of the subject. In a still more particular embodiment thereof, the pharmacological agents and/or optogenetic tools are targeted specifically to patches of cortical malformation wherein the E/I ratio is dysregulated. In a more particular embodiment, the patches of cortical malformation are located in the S1DZ. As described herein, pharmacological agents and/or optogenetic or chemogenetic tools may be administered or used alone or in conjunction with agents that modulate (promote or inhibit) IL-17 activity or interferon-gamma (IFN-γ) activity. Agents that promote Interleukin-17 (IL-17) activity include exogenous IL-17a and/or IL-17f (e.g., synthetic/manmade/recombinant IL-17a or IL-17f) and IL-17 receptor (IL-17R) agonists. Inhibitors of IL-17 activity include, e.g., antagonistic IL-17 antibodies and/or inhibitors of IL-17, IL-17R, or RORγt activity (e.g., small molecule inhibitors) and IL-17R antagonists. Agents that promote IFN-γ activity include exogenous IFN-γ (e.g., synthetic/manmade/recombinant IFN-γ) and IFN-γ receptor (IFN-γR) agonists. Agents that inhibit IFN-γ activity include, e.g., antagonistic IFN-γ antibodies, inhibitors of IFN-γ activity, small molecule inhibitors of IFN-γ, and IFN-γR antagonists.

Further to the above, a method for treating a subject afflicted with or at risk for developing a psychiatric disorder (e.g., ASD, of which autism is a particular example) that calls for selecting a subject afflicted with or at risk for developing the psychiatric disorder based at least in part on identifying a subject with cortical patches wherein the E/I ratio is dysregulated and treating the subject by administering pharmacological agents such as, for example, GABAergic receptor agonists or implementing optogenetic tools or chemogenetic tools to correct dysregulated neuronal E/I ratios in cortical patches of the subject wherein the E/I ratio is dysregulated. In a particular embodiment, the cortical patches are observed/detected in the S1DZ. In another embodiment thereof, the pharmacological agents and/or optogenetic tools are administered to the cortex of the subject to at least partially restore a normal E/I ratio (comparable to wildtype E/I ratio) in patches of cortical malformation wherein the E/I ratio is dysregulated absent treatment. In a more particular embodiment thereof, the pharmacological agents and/or optogenetic tools are administered to and/or in the vicinity of the S1DZ of the subject. In a still more particular embodiment thereof, the pharmacological agents and/or optogenetic tools are targeted specifically to patches of cortical malformation wherein the E/I ratio is dysregulated. In a more particular embodiment, the patches of cortical malformation are located in the S1DZ. As described herein, pharmacological agents and/or optogenetic or chemogenetic tools may be administered or used alone or in conjunction with agents that modulate (promote or inhibit) IL-17 activity or IFN-γ activity. Agents that promote Interleukin-17 (IL-17) activity include exogenous IL-17a and/or IL-17f (e.g., synthetic/manmade/recombinant IL-17a or IL-17f) and IL-17 receptor (IL-17R) agonists. Inhibitors of IL-17 activity include, e.g., antagonistic IL-17 antibodies and/or inhibitors of IL-17, IL-17R, or RORγt activity (e.g., small molecule inhibitors) and IL-17R antagonists. Agents that promote IFN-γ activity include exogenous IFN-γ (e.g., synthetic/manmade/recombinant IFN-γ) and IFN-γ receptor (IFN-γR) agonists. Agents that inhibit IFN-γ activity include, e.g., antagonistic IFN-γ antibodies, inhibitors of IFN-γ activity, small molecule inhibitors of IFN-γ, and IFN-γR antagonists.

Devices and methods that can be used to visualize dysregulated neuronal E/I ratios in cortical patches of a subject are described herein and known in the art and include, without limitation, functional magnetic resonance imaging (fMRI) and/or electroencephalography (EEG). Identification of autistic subjects (e.g., human patients) with dysregulated neuronal E/I ratios in the S1DZ characterizes these subjects as particularly well suited to therapeutic methods described herein. Autistic subjects in whom dysregulated neuronal E/I ratios are detected in the S1DZ are predicted to respond well to methods described herein that are designed to modulate locally the neural activity of the S1DZ (or a counterpart thereof depending on the species) using optogenetic or chemogenetic tools that are activated/implemented in a localized fashion or via localized administration of IL-17 receptor modulators to the S1DZ.

Accordingly, the invention relates to the use and application of pharmacological compounds or agents to improve or correct dysregulated neuronal E/I ratios in the cortex (in, e.g., the S1DZ) or use and implemention of optogenetic or chemogenetic tools to improve or correct dysregulated neuronal E/I ratios in the cortex for treating subjects afflicted with a psychiatric disorder or at risk for developing same. In a further aspect, the invention relates to pharmacological compounds or agents to improve or correct dysregulated neuronal E/I ratios in the cortex or use and implemention of optogenetic or chemogenetic tools to improve or correct dysregulated neuronal E/I ratios in the cortex for treating subjects afflicted with a psychiatric disorder (such as ASD) or at risk for developing same and use of such compounds or agents or optogenetic or chemogenetic tools in the preparation of a medicament for treating or preventing a psychiatric disorder. In a particular embodiment of each of these uses and applications, delivery to the cortex, the S1DZ, and/or localized delivery to cortical patches (e.g., those in the S1DZ) exhibiting dysregulated E/I ratios is envisioned.

Also envisioned herein, is a method for treating a subject afflicted with or at risk for developing a psychiatric disorder (e.g., ASD, of which autism is a particular example) that calls for selecting a subject afflicted with or at risk for developing the psychiatric disorder based in part on previous in utero exposure to MIA and/or identified as having cortical patches wherein the E/I ratio is dysregulated (at least some of which are located in the S1DZ) and treating the subject (i.e., offspring exposed to prenatal MIA) by administering agents that modulate IL-17 activity and/or IFN-γ activity. Such agents include: agents that promote IL-17 activity, including exogenous IL-17a and/or IL-17f (e.g., synthetic/manmade/recombinant IL-17a or IL-170 and IL-17R agonists; inhibitors of IL-17 activity (e.g., antagonistic IL-17 antibodies) and/or inhibitors of IL-17, IL-17R, or RORγt activity, and IL-17R; agents that promote IFN-γ activity, including exogenous IFN-γ (e.g., synthetic/manmade/recombinant IFN-γ) and IFN-γ receptor (IFN-γR) agonists; agents that inhibit IFN-γ activity including, e.g., antagonistic IFN-γ antibodies, inhibitors of IFN-γ activity, small molecule inhibitors of IFN-γ, and IFN-γR antagonists. Agents that modulate IL-17 activity or IFN-γ activity may be delivered to the cortex, the S1DZ, and/or targeted specifically to patches of cortical malformation (e.g., those in the S1DZ) that exhibit dysregulated E/I ratios.

The invention, therefore, relates to the use and application of compounds or agents that modulate IL-17 activity and/or IFN-γ activity to improve or correct dysregulated neuronal E/I ratios of the cortex for treating subjects afflicted with a psychiatric disorder or at risk for developing same. In a further aspect, the invention relates to compounds or agents that modulate IL-17 activity and/or IFN-γ activity to improve or correct dysregulated neuronal E/I ratios of the cortex for treating subjects afflicted with a psychiatric disorder (such as ASD) or at risk for developing same and use of such compounds or agents in the preparation of a medicament for treating or preventing a psychiatric disorder.

Also envisioned herein are non-invasive techniques that may be used to modulate neuronal activity including those described by McDannold et al. (2015, Sci Rep 5:16253), Park et al. (2015, Nature Biotech 33:1280), and Chuong et al. (2014, Nat Neuro 17:1123), the entire content of each of which is incorporated herein by reference.

Results presented herein also establish a developmental window of gestation wherein and/or by which time the occurrence of MIA becomes particularly significant and predictive of the generation of offspring having ASD and ASD-like phenotypes in mammalian subjects. Cross species comparisons of gestational brain development are known to those skilled in the art and such reasoning is routinely applied to extrapolate results presented in one animal model to other mammalian species, including primate species, and further including human subjects. See, for example, Clancy et al. (2007, Neurotoxicology 28:931-937) and Clancy et al. (2007, Neuroinformatics 5:79-94). Skilled persons appreciate that mouse gestation 12.5 day is analogous to the late first trimester or the second trimester of a pregnancy in a mammal and more particularly, in a human.

In sum and in accordance with the animal studies presented herein, the present inventors propose the following in order to mitigate maternal immune induced ASD and ASD-like phenotypes in mammalian subjects (e.g., in human patients and in animal models of ASD or schizophrenia): 1) Identify pregnant mothers with high risk—who are likely to produce autistic/schizophrenic children—by measuring, e.g., IL-17a levels in their sera; and 2) treat offspring (born to mothers identified as having elevated levels of IL-17a in their sera, particularly when the elevated levels were observed during the late first trimester or second trimester of the pregnancy) who exhibit ASD or ASD-like phenotypes with pharmacological agents (such as GABAergic receptor agonists) or optogenetic tools or chemogenetic tools (or other non-invasive neural activity modulating techniques) to correct dysregulated neuronal E/I ratios, particularly when found in the S1DZ. The present inventors also envison treating subjects who exhibit ASD or ASD-like phenotypes, but for whom little or no information is available regarding potential for in utero exposure to MIA, with pharmacological agents (such as GABAergic receptor agonists) or optogenetic or chemogenetic tools (or other non-invasive neural activity modulating techniques) to correct dysregulated neuronal E/I ratios, particularly when found in the S1DZ. In a particular embodiment, the treatment regimen delivers targeted therapy to the cortex of the subject. In a more particular embodiment, the treatment regimen delivers targeted therapy to the S1DZ or in the vicinity of the S1DZ of the subject. In another embodiment, the treatment regimen delivers targeted therapy to the cortical malformations where aberrant E/I ratios are observed.

The aforementioned pharmacological agonists, optogenetic tools, and/or chemogenetic tools may be administered or used alone or in conjunction with agents that modulate IL-17 activity, including: agonists or inhibitors of IL-17, agonists or inhibitors of IL-17R, and agonists or inhibitors of RORγt activity. Also envisioned herein are agents that modulate IFN-γ activity, including agonists or inhibitors of IFN-γ and agonists or inhibitors of IFN-γ receptors (IFN-γR). See, for example, Filiano et al. (2016, Nature 535:425-429; the entire content of which is incorporated herein by reference), which reveals that IFN-γ increases inhibitory neuronal activity and increases GABAergic currents. In similar fashion to agents that modulate IL-17 activity, agents that modulate IFN-γ activity may be used in conjunction with the aforementioned pharmacological agonists, optogenetic tools, and/or chemogenetic tools. It is, furthermore, envisioned that the aforementioned pharmacological agonists, optogenetic tools, and/or chemogenetic tools may be used in conjunction with agents that modulate IL-17 activity and/or agents that modulate IFN-γ activity.

It is also envisioned that agents that modulate IL-17 activity (e.g agonists or inhibitors of IL-17, agonists or inhibitors of IL-17R, and agonists or inhibitors of RORγt activity) may be used as the primary therapeutic agents in methods for treating the offspring of pregnant mothers in whom high IL-17a levels were detected in sera (particularly when the elevated levels were observed in the late first trimester or the second trimester of the pregnancy) or in autistic subjects wherein dysregulated E/I ratios are detected in the S1DZ. In such scenarios, at least one agent that modulates IL-17 activity would be administered to a subject (e.g., offspring) in need thereof. In a particular embodiment, the at least one agent that modulates IL-17 activity is administered in a localized fashion to the S1DZ. In another embodiment thereof, at least one agent that modulates IFN-γ activity is administered to a subject (e.g., offspring) in need thereof. In a more particular embodiment thereof, at least one agent that modulates IFN-γ activity is administered in a localized fashion to the S1DZ. In yet another embodiment, the at least one agent that modulates IL-17 activity is administered in conjunction with the at least one agent that modulates IFN-γ activity.

It will be understood that offspring of mothers identified as having elevated levels of IL-17a in their sera who exhibit ASD-like phenotypes, subjects in general who exhibit ASD-like phenotypes (e.g., those for whom little or no information is available pertaining to potential exposure to MIA), and subjects who exhibit ASD-like phenotypes in whom dysregulated E/I ratios are detected in their S1DZs can be treated as described herein at any age. Exemplary such ages include: infants, children, adolescents, and adults with respect to human subjects, and equivalent stages with respect to other mammalian subjects, such as mice, rats, and primates.

Other objects and advantages will become apparent to those skilled in the art from a review of the following description which proceeds with reference to the following illustrative drawings.

### BRIEF DESCRIPTION OF THE DRAWINGS

FIG. 1A-G. Cortical patches observed in the offspring of pregnant dams following MIA. A, Representative image of adult brains, stained for SATB2 (a marker for cortical neurons, Red) and DAPI (nuclear counterstain, blue) of an offspring from a poly(I:C)-treated mother. Arrow indicates the cortical patch. Scale bar represents 500 μm. B, The prevalence of cortical patches found in MIA offspring as in FIGS. 5 and 6 (N=10) plotted against cortical sub-regions and AP levels. C, Superimposed image of the cortical patches detected within AP 0.38˜−1.34 mm from individual MIA offspring as in FIG. 7 (N=50 animals). The color-coded scale (high=red; low=dark blue) indicates the differential frequencies of patches along the cortex. D, Representative images of 51 (layer I and II/III) stained for SATB2, PV, VIP or NeuN (green) in offspring from PBS- or Poly(I:C)-injected dams. Brain slices are counterstained with DAPI (blue). White dotted line indicates the boundary of cortical patches in MIA offspring. Scale bar represents 100 μm. E, Quantification of SATB2, PV, VIP and NeuN positive cells in regions centered on 51 cortical patches, divided into ten equal bins representing different depths of the cortex, in MIA offspring or in corresponding regions in PBS offspring, (n=4 PBS and 4 Poly(I:C) for SATB2; n=6 PBS and 10 Poly(I:C) for PV; n=4 PBS and 4 Poly(I:C) for VIP; n=4 PBS and 4 Poly(I:C) for NeuN; from four to five independent experiments). F, Representative images of c-Fos expression in 51 of PBS (PBS) or MIA (Poly(I:C)) offspring. Scale bar represents 100 μm. G, Quantification of c-Fos positive cells (n=5 PBS and 4 Poly(I:C); from four independent experiments). *p<0.05, **p<0.01 as calculated by two-way ANOVA with Sidak post-hoc tests (E) and Student's t-test (G). Graphs indicate mean±s.e.m.

FIG. 2A-E. The presence and size of cortical patches are predictive of MIA-induced behaviors and their severity in offspring. A, Top: Representative images of SATB2 and TBR1 staining in the adult brains of offspring from pregnant dams injected with PBS or poly(I:C) at embryonic stages E12.5, E15.5, or E18.5. Bottom: Percentage of offspring with cortical patches in S1 upon maternal administration of poly(I:C). White arrows indicate the cortical patch. Scale bar represents 100 μm. B-E, The cortical patch size is plotted against the severity of the featured MIA phenotypes on the marble burying test (B), sociability test (C), and open field test (D) or against the total distance moved during the sociability test (E) for offspring exposed to MIA at E12.5 (green) or E15.5 (red). R² was calculated with results from the E12.5 group (n=18 (E12.5) and n=15 (E15.5); from five to seven independent experiments). Green lines represent the slope of 95% confidence intervals for the E12.5 group.

FIG. 3A-Q. Increasing neural activity in S1 centered on S1DZ recreates MIA behavioral phenotypes in WT mice. A, Schematic (left) and a representative image (right) of the brain injected with EYFP-expressing virus and implanted with optical fibers in the cortical region centered on the S1DZ. Scale bar represents 1 mm. B, Optical stimulation protocol. Light was delivered in 3-min intervals for a total duration of 18 min in the marble burying test and of 9 min in the sociability and open field tests. Animals started either with a Laser On or Off session in a counterbalanced manner. C, A schematic of labeled cells in PBS offspring infected with viruses (green) driving EYFP, ChR2 or NpHR under the neuron specific promoter hSyn (Pyr: Pyramidal neuron, PV: Parvalbumin-positive neuron, and Ast: Astrocyte). D-G, The marble burying index (the percentage of marbles buried during the 18-min behavioral session) (D), the sociability index (the percentage of time spent investigating the social or inanimate stimulus out of the total exploration time for both objects during the 1^{st} laser-on session of the sociability test) (E), the total interaction time (the total exploration time for both objects during the 1^{st} laser-on session of the sociability test) (F), and the time spent in the center during the 1^{st} laser-on session of the open field test (G) for animals prepared as in (C) (n=12, 12, and 18 for PBS offspring injected with AAV₂-hSyn-EYFP, ChR2, or NpHR from seven to eight independent experiments). H, A schematic of the labeled cells (green) in vGluT2-Cre mice injected with viruses driving EYFP, ChR or NpHR in a Cre-dependent manner. I-L, Performance on the marble burying (I) and the sociability (J) tests, the total interaction time during the sociability test (K), and the time spent in the center during the open field assay (L) for animals prepared as in (H) (n=10, 17, and 11 for animals injected with AAV₂-EF1a-DIO-EYFP, ChR2, or NpHR from seven to eight independent experiments). M, A schematic of the labeled cells (green) in PV-Cre mice injected with viruses driving EYFP, ChR2 or NpHR in a Cre-dependent manner. N-Q, Performance on the marble burying (N) and the sociability (0) tests, the total interaction time during the sociability test (P), and the time spent in the center during the open field assay (Q) for animals prepared as in (M) (n=13, 12, and 15 for animals injected with AAV₂-EF1a-DIO-EYFP, ChR2, or NpHR from seven to eight independent experiments). *p<0.05, **p<0.01 as calculated by two-way ANOVA with Sidak post-hoc tests (E, J, O) and one-way ANOVA with Tukey post-hoc tests (D,F,G,I,K,L,N,P,Q). Graphs indicate mean±s.e.m.

FIG. 4A-M. Reducing neural activity in S1DZ corrects behavioral abnormalities in MIA offspring. A, Representative images of c-Fos expression upon photostimulation of the S1DZ in MIA offspring, in which AAV₂-hSyn-EYFP, ChR2-EYFP, or NpHR-EYFP is injected into the S1DZ. Coronal sections of the brains were stained for c-Fos (red) and EYFP (green), and counterstained with neurotrace (NT, blue). Scale bar represents 100 μm. B, The percentage of EYFP⁺ neurons co-expressing c-Fos upon photostimulation of the injection sites of the animals described in (A). C-F, Performance on the marble burying (C) and the sociability (D) tests, the total interaction time during the sociability test (E), and the time spent in the center during the open field assay (F) for animals prepared as in (A) (n=8 for PBS offspring injected with AAV₂-hSyn-EYFP; n=11, 10, and 20 for MIA offspring injected with AAV₂-hSyn-EYFP, ChR2, or NpHR from seven to nine independent experiments). G, Projection profiles of anterograde tracing using AAV₂-hSyn-mCherry (red) in the S1FL and AAV₂-hSyn-EYFP (green) in the S1DZ (TeA: Temporal association cortex, ECT: Ectorhinal cortex and M2: Secondary motor cortex). Scale bar represents 300 μm. H, Projection profiles of anterograde tracing using AAV₂-hSyn-mCherry (red) in the S1BF and AAV₂-hSyn-EYFP (green) in the S1DZ. Scale bar represents 300 μm. I, Schematic showing the rabies virus injection sites into TeA and the optic fiber implantation sites in S1DZ. J, Staining for EYFP (green), c-Fos (red) and DAPI (blue) of the fiber implantation site after photostimulation of the animals described in (I). Scale bar represents 300 μm. K-M, Performance on the marble burying (K) and the sociability (L) tests, and the total interaction time during the sociability test (M) for animals prepared as in (I) (n=8, 13, and 10 for PBS offspring injected with RV-EYFP, Chronos, or ArchT; n=12 for MIA offspring injected with RV-ArchT from five to seven independent experiments). *p<0.05, **p<0.01 as calculated by two-way ANOVA with Sidak post-hoc tests (D,L) and one-way ANOVA with Tukey post-hoc tests (B,C,E,F,K,M). Graphs indicate mean±s.e.m.

FIG. 5. Distribution of cortical patches in the cortex of MIA offspring. The locations of the cortical patches of 10 individual MIA animals were matched to their corresponding AP levels in the Paxinos brain atlas. Different colors represent the patches from different mice. The sub-regions, in which the cortical patches were observed in more than 3 or 5 animals, are circled in blue or red, respectively. PrL: Prelimbic, MO: Medial orbital, DLO: Dorsolateral orbital, DI: Dysgranular insular, FrA: Frontal association cortex, M1: Primary motor cortex, M2: Secondary motor cortex, 51: Primary somatosensory cortex, S2: Secondary somatosensory cortex, V1: Primary visual cortex, V2: Secondary visual cortex, AU1: Primary auditory cortex, AUD: Secondary auditory cortex, dorsal area, AUV: Secondary auditory cortex, ventral area, Cg/RS: Cingulate/Retrosplenial cortex.

FIG. 6A-C. Distribution of cortical patches in the brains of MIA offspring according to cortical sub-region and AP levels. A, Prevalence of cortical patches in different cortical sub-regions of the MIA offspring described in FIG. 5 (n=10). B, Prevalence of cortical patches at different AP levels of the brain in the MIA offspring described in FIG. 5 (n=10). Individual AP levels correspond to those in the schematic images of FIG. 5. C. The size and frequencies of cortical patches found in different cortical sub-regions of the MIA offspring described in FIG. 5 (n=10). FrA: Frontal association cortex, M1: Primary motor cortex, M2: Secondary motor cortex, S1: Primary somatosensory cortex, S2: Secondary somatosensory cortex, V1: Primary visual cortex, V2: Secondary visual cortex, AUD: Secondary auditory cortex, dorsal area, AU1: Primary auditory cortex, AUV: Secondary auditory cortex, ventral area, Cg/RS: Cingulate/Retrosplenial cortex, and TeA: Temporal association cortex. *p<0.05, **p<0.01 as calculated by one-way ANOVA with Tukey post-hoc tests (C). Graphs indicate mean±s.e.m.

FIG. 7A-B. Distribution of cortical patches located within 0.38˜−1.34 AP in the brains of MIA offspring. A, Schematics of the cortical patches located within 0.38˜−1.34 AP in the brains of MIA offspring plotted onto the atlas plane near ˜−0.5 AP. The size of the cortical patches in the schematic is scaled to reflect the actual size as accurately as possible. Blue indicates the cortical patches from one hemisphere and red from the other. B, Representative images of the cortical patches in the brains of MIA offspring (A) stained with TBR1 or SATB2 and counterstained with DAPI. The number, locations and sizes of the cortical patches observed at a given AP level along with each animal's behavioral performance on the marble burying (marble burying index), sociability (% interaction) and the time spent in the center (s) of an open field are indicated. White arrows indicate cortical patches. Scale bar represents 300 μm.

FIG. 8A-C. MIA offspring display reduced inhibitory drive onto pyramidal neurons in S1 cortical patches. A, Representative traces of mIPSCs from 51 layer II/III pyramidal neurons of PBS offspring and from 51 cortical patches of MIA offspring. B-C, Average population data depicting the frequency (B) and amplitude (C) of pharmacologically isolated mIPSCs in layer II/III pyramidal neurons (n=13 and 10 from PBS and MIA offspring, respectively). *p<0.05 as calculated by Student's t-test (B,C). Graphs indicate mean±s.e.m.

FIG. 9A-L. The development of MIA-associated behaviors depends on the time point at which MIA is induced. A-C, Schematics of the marble burying test (A), sociability test (B), and open field test (C). D, The ultrasonic vocalization (USV) index represents the number of USVs made by the pups (n=20, 24, 21, and 22 for PBS, E12.5, E15.5, or E18.5 groups from five to seven independent experiments). E-I, The marble burying index (the percentage of marbles buried during the 15-min marble burying test) (E), the sociability index (% interaction or the percentage of time spent investigating the social or inanimate stimulus out of the total exploration time of both objects during the 10-min sociability test) (F), the total interaction time (the total exploration time of both objects during the 10-min sociability test) (G), the total distance moved (within the 3-chamber arena during the 10-min sociability test) (H), and the time spent in the center of an open field (during the 15-min open field test) (I) of the adult offspring described in (D). (n=12, 18, 15, and 9 for PBS, E12.5, E15.5, or E18.5 groups from five to seven independent experiments). J-L The size of cortical patches found outside of S1 in offspring from dams injected with poly(I:C) at E12.5 is plotted against the severity of the featured MIA phenotypes on the marble burying test (J), sociability test (K), and open field test (L). Lines represent the slope of 95% confidence intervals (n=18 from E12.5 group, seven to nine independent experiments). *p<0.05, **p<0.01 as calculated by two-way ANOVA with Sidak post-hoc tests (F), one-way ANOVA with Tukey post-hoc tests (D,E,G,H,I), and Linear regression (J,K,L). Graphs indicate mean±s.e.m.

FIG. 10A-B. IL-17Ra expression is required in the fetal brain to induce behavioral abnormalities upon MIA. A, Schematic showing the breeding scheme. Homozygous IL-17Ra KO animals carrying Nestin-Cre transgene was crossed to homozygous IL-17Ra conditional line (IL-17Ra^{fl/fl}). B, Representative images of SATB2 (red) staining in S1 of offspring with indicated genotypes (WT, IL-17Ra^{fl}/KO, or IL-17Ra^{fl}/KO;Cre) from mothers injected with PBS or poly(I:C). Scale bar represents 100 μm.

FIG. 11A-F. Increasing neural activity in WT animals induces MIA behavioral phenotypes. A, Representative images of c-Fos expression upon photostimulation of the S1DZ in WT offspring from mothers injected with PBS at 12.5 (PBS offspring). In these animals, AAV₂-hSyn-EYFP, ChR2-EYFP, or NpHR-EYFP viruses were targeted to the S1DZ. Coronal sections of the brains were stained for c-Fos (red) and EYFP (green), and counterstained with neurotrace (NT, blue). Scale bar represents 100 μm. B, The percentage of EYFP⁺ neurons expressing c-Fos upon photostimulation of the injection sites in animals as prepared in (A). C-F, The marble burying index (C), the social preference index (D), the total interaction time during the sociability assay (E), and the time spent in the center during the open field assay (F) are plotted as averages from each individual 3-min sessions. Light blue indicates the laser ‘On’ sessions (Laser On-Off: n=6, 5, and 8; ‘Laser Off-On’: n=6, 7, and 10 from the AAV₂-hSyn-EYFP, ChR2-EYFP, or NpHR-EYFP injected PBS offspring). *p<0.05, **p<0.01 as calculated by two-way ANOVA with Sidak post-hoc tests (C,D,E,F) and one-way ANOVA with Tukey post-hoc tests (B). Graphs indicate mean±s.e.m.

FIG. 12A-F. Increasing neural activity in the vGluT2-positive neurons of WT animals creates MIA behavioral phenotypes. A, Representative images of c-Fos expression upon photostimulation of the S1DZ in vGluT2-Cre animals, in which AAV₂-EF1a-DIO-EYFP, ChR2-EYFP, or NpHR-EYFP viruses were targeted to the S1DZ. Coronal sections of the brains were stained for c-Fos (red) and EYFP (green), and counterstained with neurotrace (NT, blue). Scale bar represents 100 μm. B, The percentage of EYFP⁺ neurons expressing c-Fos upon photostimulation of the injection sites in animals as prepared in (A). C-F, The marble burying index (C), the social preference index (D), the total interaction time during the sociability assay (E), and the time spent in the center during the open field assay (F) are plotted as averages from each 3-min sessions. Light blue indicates the laser ‘On’ sessions (Laser On-Off: n=5, 8, and 6; ‘Laser Off-On’: n=5, 9, and 5 from the AAV₂-EF1a-DIO-EYFP, ChR2-EYFP, or NpHR-EYFP injected vGluT2-Cre animals). *p<0.05, **p<0.01 as calculated by two-way ANOVA with Sidak post-hoc tests (C,D,E,F) and one-way ANOVA with Tukey post-hoc tests (B). Graphs indicate mean±s.e.m.

FIG. 13A-F. Decreasing neural activity in the PV-positive neurons of WT animals creates MIA behavioral phenotypes. A, Representative images of c-Fos expression upon photostimulation of the S1DZ in PV-Cre animals injected with AAV₂-EF1α-DIO-EYFP, ChR2-EYFP, or NpHR-EYFP viruses. Coronal sections of the brains were stained for c-Fos (red) and EYFP (green), and counterstained with neurotrace (NT, blue). Scale bar represents 100 μm. B, The percentage of EYFP⁺ neurons expressing c-Fos upon photostimulation of the injection sites in animals as prepared in (a). C-F, The marble burying index (C), the social preference index (D), the total interaction time during the sociability assay (E), and the time spent in the center during the open field assay (F) are plotted as averages for each 3-min sessions. Light blue indicates the laser ‘On’ sessions (Laser On-Off: n=7, 6, and 8; ‘Laser Off-On’: n=6, 6, and 7 from the AAV₂-EF1a-DIO-EYFP, ChR2-EYFP, or NpHR-EYFP injected PV-Cre animals). *p<0.05, **p<0.01 as calculated by two-way ANOVA with Sidak post-hoc tests (C,D,E,F) and one-way ANOVA with Tukey post-hoc tests (B). Graphs indicate mean±s.e.m.

FIG. 14A-F. The ability to create MIA behavioral phenotypes by increasing neural activity in the primary somatosensory cortex of WT animals is specific with respect to the AP level. A, Schematics (top) and representative images (bottom) of the five sites in the 51 of WT animals injected with either AAV₂-hSyn-EYFP or AAV₂-hSyn-ChR2-EYFP virus (green) (AP=+0.5, +0.0, −0.5, −1.0, or −1.5 mm). S1HL: Primary somatosensory, hindlimb, S1FL: Primary somatosensory, forelimb, 51: Primary somatosensory cortex, S1DZ: Primary somatosensory, dysgranular zone, S1BF: Primary somatosensory, barrel field, S1ShNc: Primary somatosensory, shoulder and neck, S1Tr: Primary somatosensory, trunk. Scale bar represents 300 μm. B, Representative images of c-Fos expression upon photostimulation of the injection sites in animals as prepared in (A). Coronal sections of the brains were stained for c-Fos (red) and EYFP (green), and counterstained with neurotrace (NT, blue). Scale bar represents 100 μm. C, The percentage of EYFP⁺ neurons expressing c-Fos upon photostimulation of the injection site. D-F, The marble burying index (the percentage of marbles buried during the 18-min behavioral sessions) (D), the sociability index (the percentage of time spent investigating the social or inanimate stimulus out of the total exploration time for both objects during the 1^{st} laser-on session) of the sociability test (E), and the total interaction time (the total exploration time of both objects during the 1^{st} laser-on session) of the sociability test (F) for animals prepared as in (A) (n=11 for WT animals injected with AAV₂-hSyn-EYFP at AP=−0.5 mm and n=12, 7, 10, 12, and 9 for those injected with AAV₂-hSyn-ChR2-EYFP at AP=0.5, 0.0, −0.5, −1.0, or -1.5 mm, respectively).*p<0.05, **p<0.01 as calculated by two-way ANOVA with Sidak post-hoc tests (E) and one-way ANOVA with Tukey post-hoc tests (c,d,f). Graphs indicate mean±s.e.m.

FIG. 15A-F. MIA behavioral phenotypes are induced in WT animals by increasing neural activity specifically in the S1DZ region. A, A schematic showing the superimposed virus injection sites from individual WT animals, in which AAV₂-hSyn-ChR2-EYFP was delivered into the S1FL (blue), S1DZ (red), or S1BF (green). B, Representative images of c-Fos expression upon photostimulation of the injection sites shown in (A). Coronal sections of the brains were stained for c-Fos (red) and EYFP (green), and counterstained with neurotrace (NT, blue). Scale bar represents 100 μm. C, The percentage of EYFP⁺ neurons co-expressing c-Fos upon photostimulation of the injection site. D-F, The marble burying index (the percentage of marbles buried during the 18-min behavioral sessions) (D), the sociability index (the percentage of time spent investigating the social or inanimate stimulus out of the total exploration time for both objects during the 1^{st} laser-on session) of the sociability test (E), and the total interaction time (the total exploration time for both objects during the 1^{st} laser-on session) of the sociability test (F) for animals prepared as in (A) (n=7 for WT animals injected with AAV₂-hSyn-EYFP into S1DZ and n=10, 12, and 10 for WT animals injected with AAV₂-hSyn-ChR2-EYFP into S1FL, S1DZ, or S1BF). *p<0.05, **p<0.01 as calculated by two-way ANOVA with Sidak post-hoc tests (E) and one-way ANOVA with Tukey post-hoc tests (C,D,F). Graphs indicate mean±s.e.m.

FIG. 16A-D. Reducing neural activity in the cortical region centered on the S1DZ corrects the behavioral abnormalities of MIA offspring. A-D, The marble burying index (A), the social preference index (B), the total interaction time during the sociability assay (C), and the time spent in the center during the open field assay (D) are potted as averages for each 3-min sessions. AAV₂-hSyn-EYFP, ChR2-EYFP, or NpHR-EYFP viruses were targeted to the S1DZ of MIA offspring (poly(I:C)). Light blue indicates the laser ‘On’ sessions (Laser On-Off: n=6, 5, and 10; ‘Laser Off-On’: n=5, 5, and 10 from the AAV₂-hSyn-EYFP, ChR2-EYFP or NpHR-EYFP injected MIA offspring). *p<0.05, **p<0.01 as calculated by two-way ANOVA with Sidak post-hoc tests (A,B,C,D). Graphs indicate mean±s.e.m.

FIG. 17. The S1FL and S1DZ exhibit distinct efferent targets. AAV₂-hSyn-mCherry and AAV₂-hSyn-EYFP were injected into the S1FL and S1DZ, respectively. The two cortical regions project to distinct sub-regions of the M2, the striatum, and the associative cortices. Representative images are aligned to their corresponding AP levels in the Paxinos brain atlas (n=4). Scale bar represents 1 mm.

FIG. 18. The S1DZ and S1BF exhibit distinct efferent targets. AAV₂-hSyn-EYFP and AAV₂-hSyn-mCherry were injected into S1DZ and S1BF, respectively. The two cortical regions project to distinct sub-regions of the M2, the striatum, and the associative cortices. Representative images are aligned to their corresponding AP levels in the Paxinos brain atlas (n=6). Scale bar represents 1 mm.

FIG. 19A-D. Altering the neural activity of the neurons in the S1DZ that project to the TeA modulates sociability without altering marble burying behavior both in WT animals and MIA offspring. A, RV-EYFP, Chronos-EYFP, or ArchT-EYFP viruses were targeted into the TeA. The percentage of EYFP⁺ neurons co-expressing c-Fos in different sub-regions of 51 upon photostimulation of the retrogradely labeled neurons of the S1DZ in WT PBS offspring (PBS) or MIA offspring (Poly(I:C)). S1HL: Primary somatosensory, hindlimb, S1FL: Primary somatosensory, forelimb, S1DZ: Primary somatosensory, dysgranular zone, and S1BF: Primary somatosensory, barrel field. B-D, The marble burying index (B), the social preference index (C), and the total interaction time during the sociability assay (D) are averages of individual 3-min sessions. Light blue indicates the laser ‘On’ sessions (Laser On-Off: n=3, 6, 6, and 7; ‘Laser Off-On’: n=5, 7, 4, and 5 for WT PBS offspring injected with RV-EYFP, Chronos-EYFP, or ArchT-EYFP, or for MIA offspring injected with RV-ArchT-EYFP). *p<0.05, **p<0.01 (a) and *p<0.05 for statistical comparison between the WT PBS offspring injected with RV-EYFP or Chronos; +p<0.05, ++p<0.01 for statistical comparison between WT PBS offspring injected with RV-EYFP and MIA offspring injected with RV-ArchT-EYFP (B-D) as calculated by two-way ANOVA with Sidak post-hoc tests (B,C,D) and one-way ANOVA with Tukey post-hoc tests (A). Graphs indicate mean±s.e.m.

FIG. 20. Schematic depicting timeline for inducing MIA and potential therapeutic intervention for offspring afflicted with ASD or an ASD-like condition as a consequence thereof.

### DETAILED DESCRIPTION

The present inventors previously reported that the offspring from pregnant dams injected with polyinosinic:polycytidylic acid (poly(I:C)), which mimics viral infection, on embryonic day 12.5 (E12.5) exhibit behavioral abnormalities including abnormal communication, increased repetitive behaviors, and deficits in sociability 11 As observed in autistic human patients¹², MIA-affected rodent offspring also displayed patches of disorganized cortical cytoarchitecture. A loss of the cortical layer-specific markers special AT-rich sequence-binding protein 2 (SATB2) and T-brain-1 (TBR1) was observed during embryonic development as well as in adulthood¹¹. Development of both MIA-associated behavioral phenotypes (MIA behaviors) and cortical patches were prevented by knocking out a key transcriptional regulator of Th17 cells, retinoic acid receptor-related orphan nuclear receptor gamma t (RORγt), in maternal T-cells, or by inhibiting the activity of their effector cytokine IL-17a in pregnant dams¹¹. These observations suggested that the maternal Th17 cell/IL-17a pathway is crucial for mediating MIA behaviors and for generating cortical patches in the offspring. These findings are detailed in U.S. application Ser. No. 15/042,976 and in Choi et al. (2016, Science 351:933-939), the entire content of each of which is incorporated herein by reference. However, whether the cortical phenotype is the underlying cause of the behavioral abnormalities in MIA offspring remained undetermined.

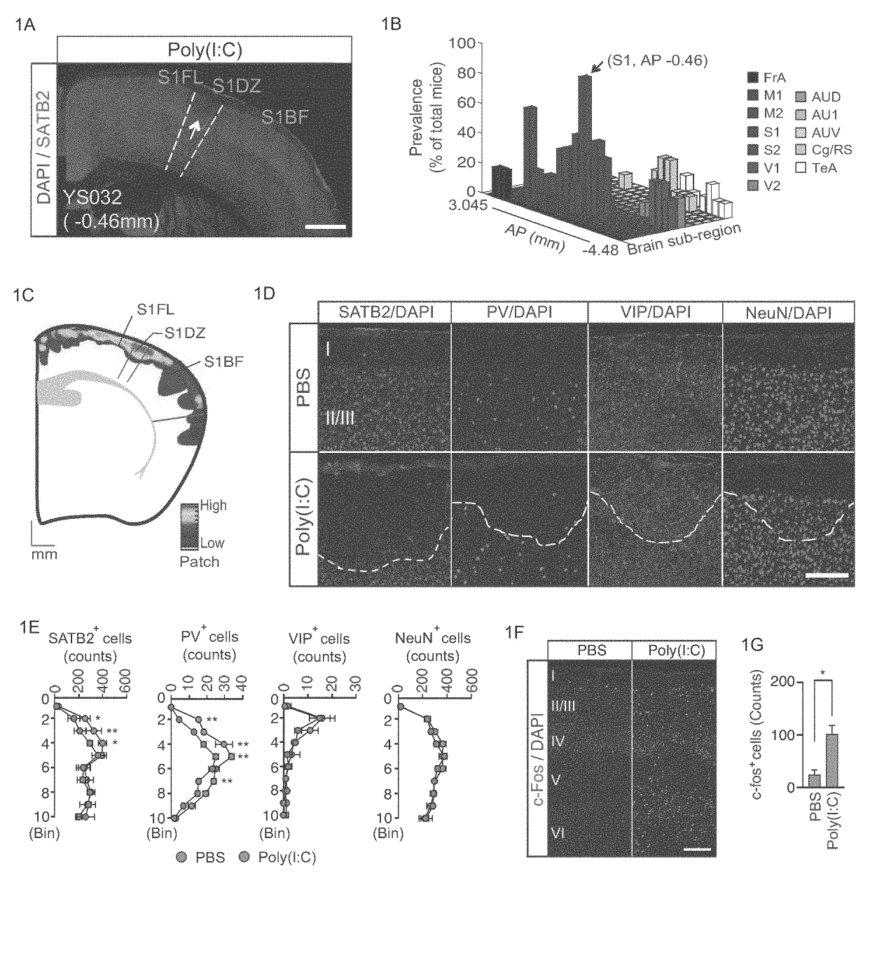

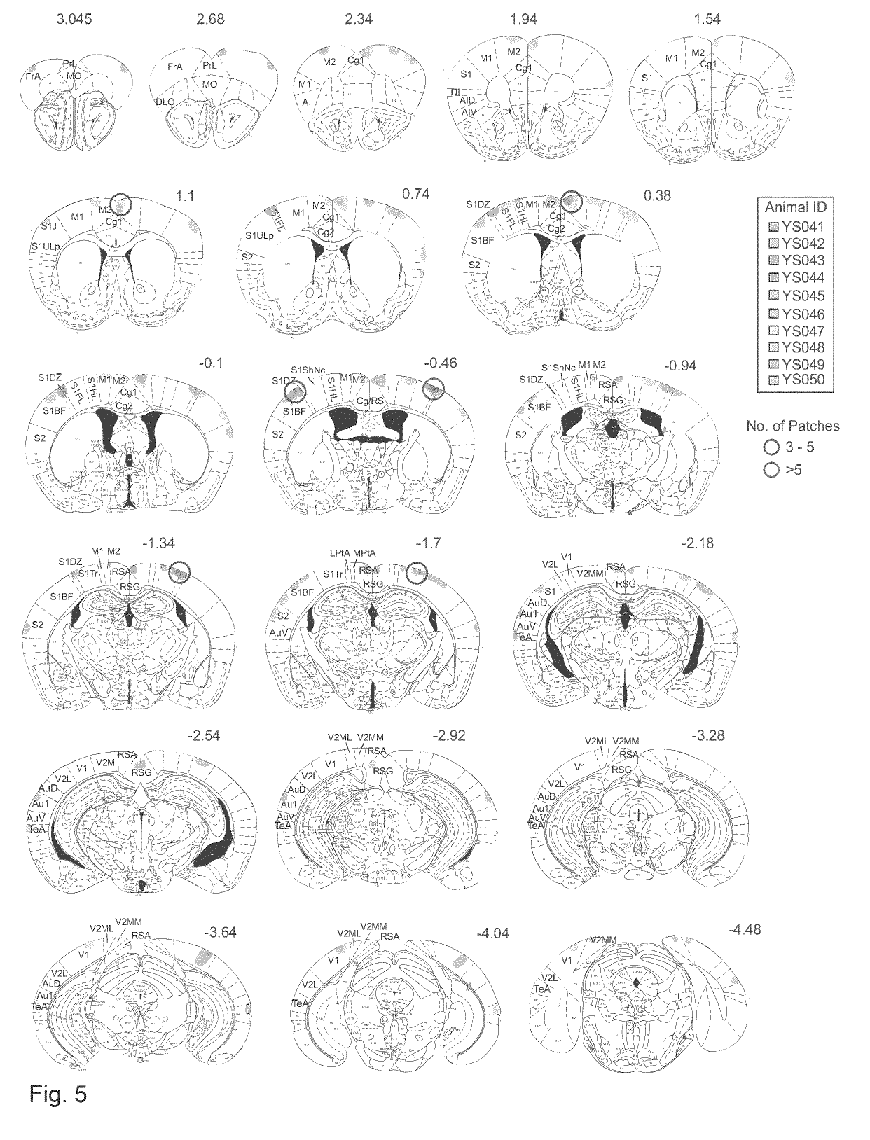

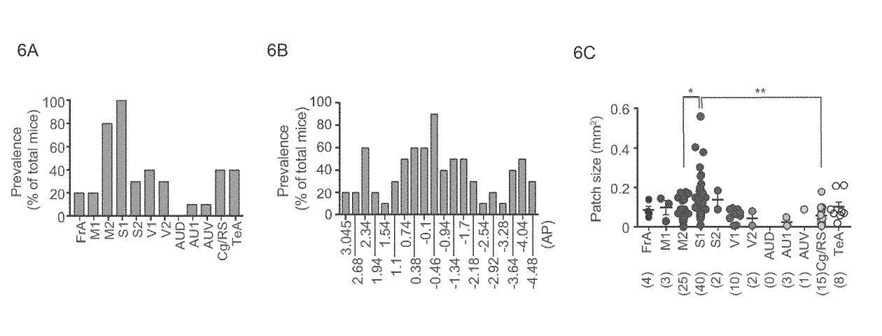

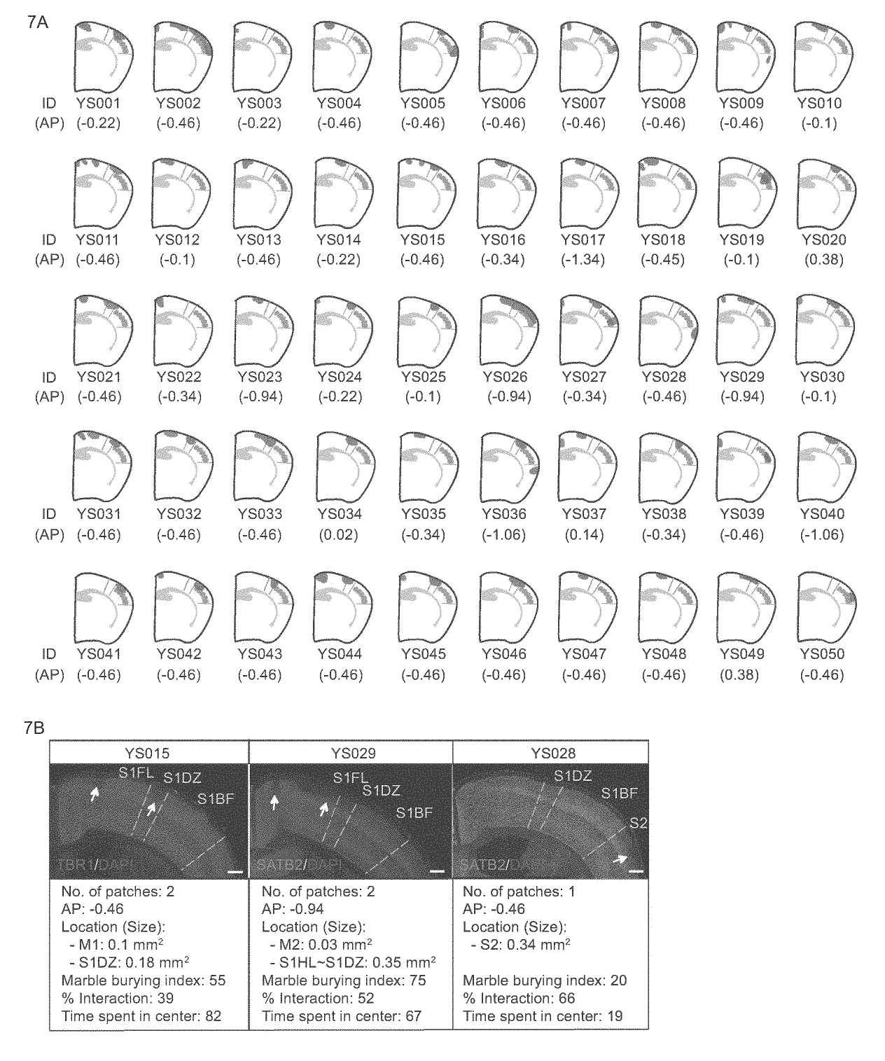

Further to the above, the present inventors first wished to determine the distribution of cortical patches in the brains of adult MIA offspring by matching the locations of cortical regions that lack expression of SATB2 or TBR1 to those in a reference mouse brain atlas (FIG. 1A)¹³. Cortical patches found in individual animals often retained similar mediolateral (ML) and dorsoventral (DV) coordinates through serial coronal sections, suggesting that many form a single continuous patch extending along the AP axis, rather than forming a series of independent patches (FIG. 5, 6). Although cortical patches were detected at multiple locations throughout the cortex, they were often prevalently observed in the primary somatosensory cortex (S1) (100% of animals, N=10) and at the anteroposterior (AP) level˜0.5 mm posterior to the Bregma (AP=−0.5 mm) (90% of animals, N=10) (FIG. 1B and FIG. 5, 6), as well as in the secondary motor cortex (M2) and other cortical regions, including the temporal association area (TeA) (80% and 40% of animals, respectively, N=10) (FIG. 1B and FIG. 6). Among these regions, cortical patches were also most predominantly present in S1 with respect to both their number and sizes (FIG. 6C). Furthermore, registration of cortical patches in individual MIA animals onto the same reference plane near ˜AP −0.5 mm revealed that the cortical patches most consistently centered on S1DZ, a region of the primary somatosensory cortex that is morphologically characterized by the absence of a discernible 4th cortical layer and functionally to muscle- and joint-related responses (56% of animals, N=50) (FIG. 1C, and FIG. 7)¹⁴⁻¹⁶. Based on these results, further analysis on S1 patches near ˜AP-0.5 mm was performed.

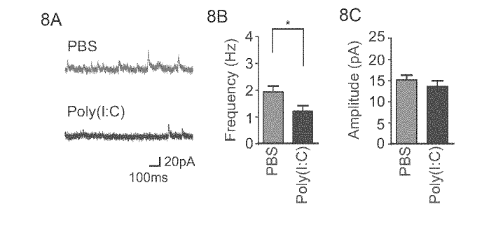

Deficits in interneuron function or dysregulation of neural activity in the somatosensory pathway have been previously associated with both genetic and environmental mouse models of autism¹⁷⁻¹⁹ On a similar note, the present inventors found that S1 cortical patches of MIA offspring display a specific loss of PV⁺ cortical neurons, which are a class of interneurons derived from the medial ganglionic eminence²⁰. The present inventors, however, observed no significant differences in the expression of the neuronal specific marker NeuN or of the vasoactive intestinal polypeptide (VIP), which is expressed in interneurons derived from the caudal ganglionic eminence, between PBS control and MIA offspring (FIG. 1D, E)^{20,21}. To test whether this selective loss of PV⁺ interneurons results in a diminished inhibitory drive onto S1 pyramidal neurons, whole-cell patch clamp recordings were used to measure the miniature Inhibitory Post-Synaptic Currents (mIPSCs) in S1 layer II/III pyramidal neurons of PBS or MIA offspring. These investigations revealed a reduction in frequency, but not amplitude, of mIPSC (FIG. 8) paralleled by an increase in the number of S1 neurons expressing c-Fos, a marker of neuronal activation (FIG. 1F,G).

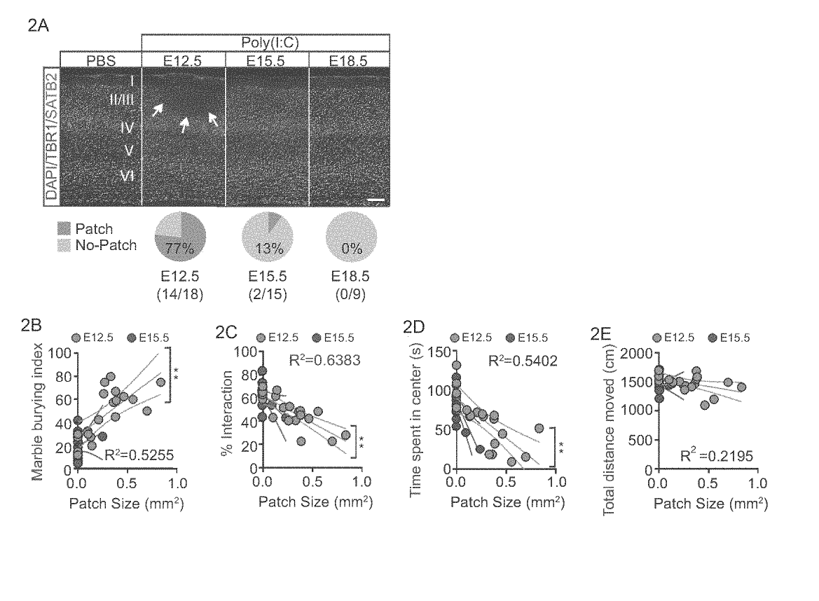

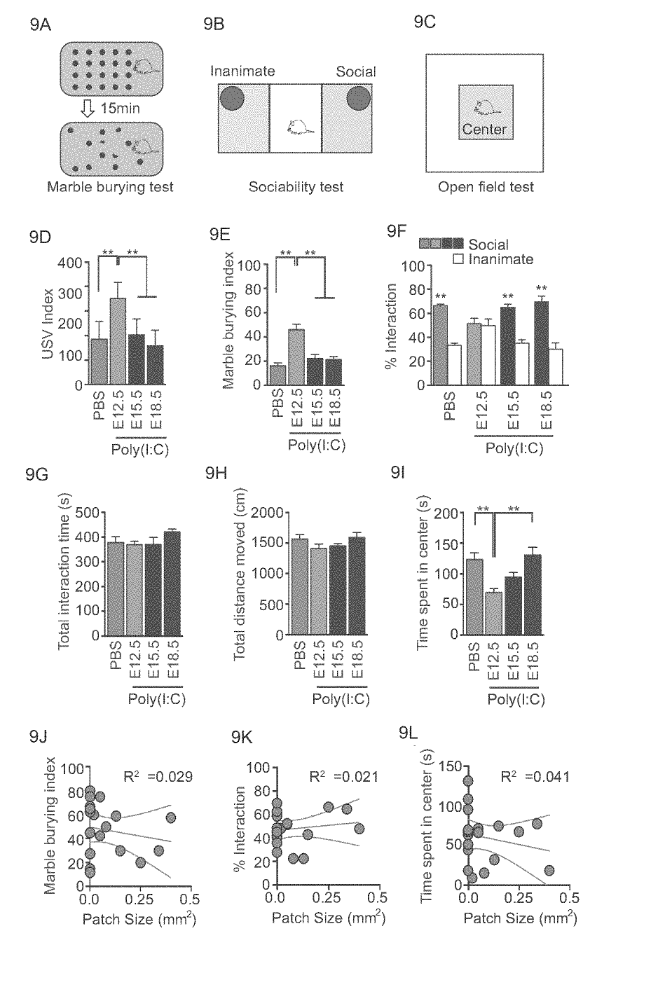

In order to determine whether the development of MIA-associated behaviors and the appearance of cortical patches depend on the developmental timing at which MIA is induced, the present inventors injected poly(I:C) into pregnant dams at embryonic stages E12.5, E15.5, or E18.5, and assessed their offspring for MIA-associated behavioral phenotypes. The present inventors first examined the offspring's ability to communicate socially by measuring the ultrasonic vocalization (USV) made by pups upon separation from their mothers at postnatal day 9 (P9). As previously reported¹¹, pups from pregnant dams injected with poly(I:C) at E12.5 (MIA offspring) emitted more USV calls than pups from PBS-injected dams (PBS offspring). However, such an increase was not observed in the offspring from dams injected with poly(I:C) either at E15.5 or E18.5 (FIG. 9D). The present inventors also examined repetitive behaviors using the marble-burying assay, natural inclination towards social targets with the sociability assay as well as anxiety-related behaviors by measuring the time spent by adult MIA offspring at the center of an open field (FIG. 9A-C). For all behaviors, deficits were observed in the offspring when exposed to prenatal MIA at E12.5. Again, the offspring's behaviors from mothers injected with poly(I:C) either at E15.5 or E18.5 were indistinguishable from those from PBS-injected mothers (FIG. 9E, F, J). Importantly, the total interaction time and the total distance traveled during the sociability assay were similar between the different treatment groups suggesting that differences in activity or arousal levels cannot explain the observed behavioral differences (FIG. 9G, H). Results presented herein strongly indicate that all behavioral abnormalities emerge from a discrete developmental stage, allowing us to examine whether the presence of cortical patches is predictive of MIA-behavioral phenotypes. Indeed cortical patches were observed in the S1 in 77% of offspring from dams injected with poly(I:C), but not PBS, at E12.5. Yet, cortical patches were seen only in 13% or none of the offspring when poly(I:C) was administered at E15.5 or E18.5, respectively (FIG. 2a). Thus, maternal-inflammation induced at E15.5 and E18.5, unlike at E12.5, was ineffective in generating cortical patches and also failed to produce behavioral abnormalities in MIA offspring. Furthermore, the size of the S1 cortical patches correlated with the severity of behavioral phenotypes: the cortical patch sizes ranged between 0 (absence of the cortical patch) and 1 mm² and were positively correlated with the marble burying index, while negatively correlated with both sociability and time spent in the center of an open field (FIG. 2b-d). The total distance traveled during the sociability assay was not affected by patch size (FIG. 2e). On the other hand, the size of cortical patches found outside of S1 did not correlate with the severity of behavioral abnormalities (FIG. 9J-L).

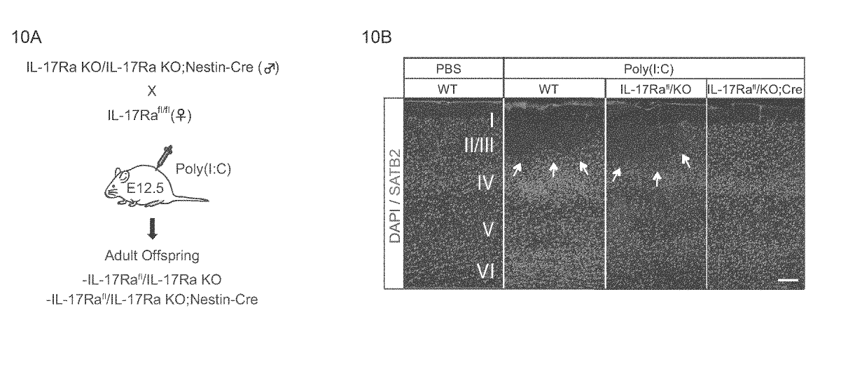

As indicated herein above, the present inventors previously showed that the ability of IL-17a to induce MIA-associated phenotypes requires intact IL-17a receptor subunit A (IL-17Ra) expression in the fetus¹¹. In line with this observation, knocking-out IL-17Ra in offspring using a Cre driver line specific for the nervous system—Nestin-Cre—prevented the development of S1 cortical patches (FIG. 10). Together, these data collectively show that the presence of cortical patches is highly predictive of MIA-induced behaviors. They further suggest that timing of inflammation, as well as IL-17Ra expression in the fetal brain dictate the severity of ASD-like behavioral phenotypes in offspring by contributing to the formation of S1 cortical patches.

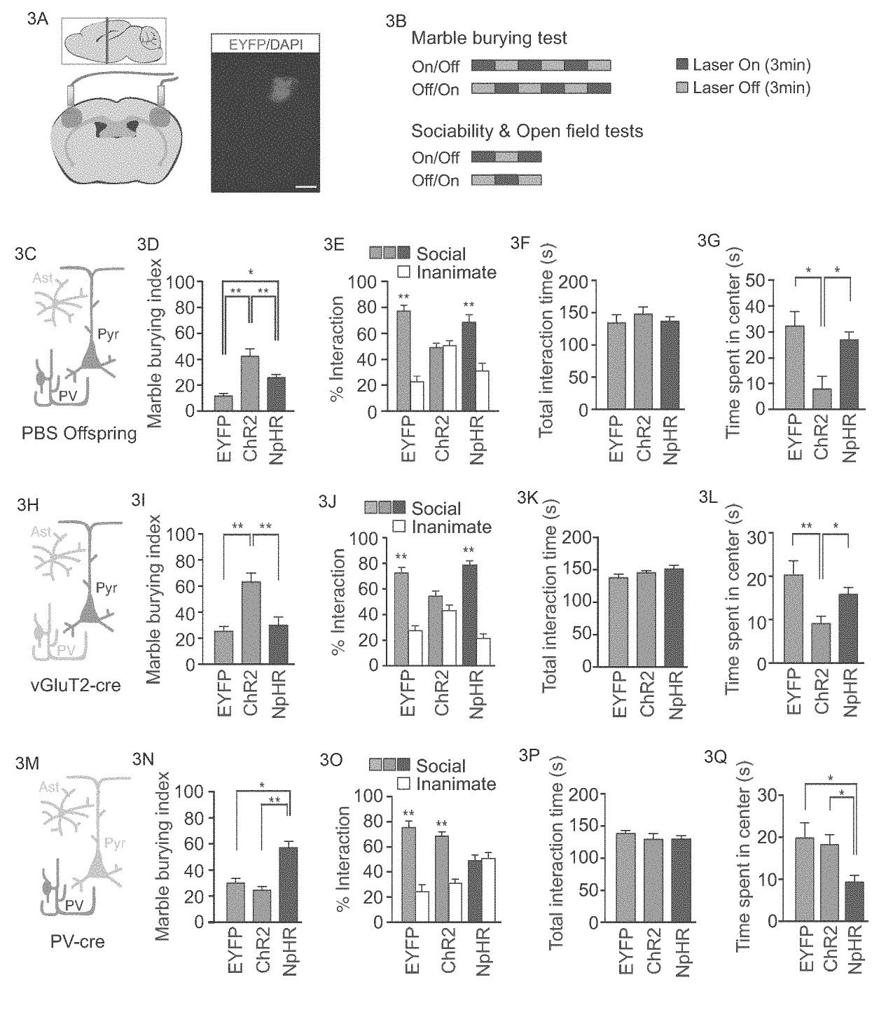

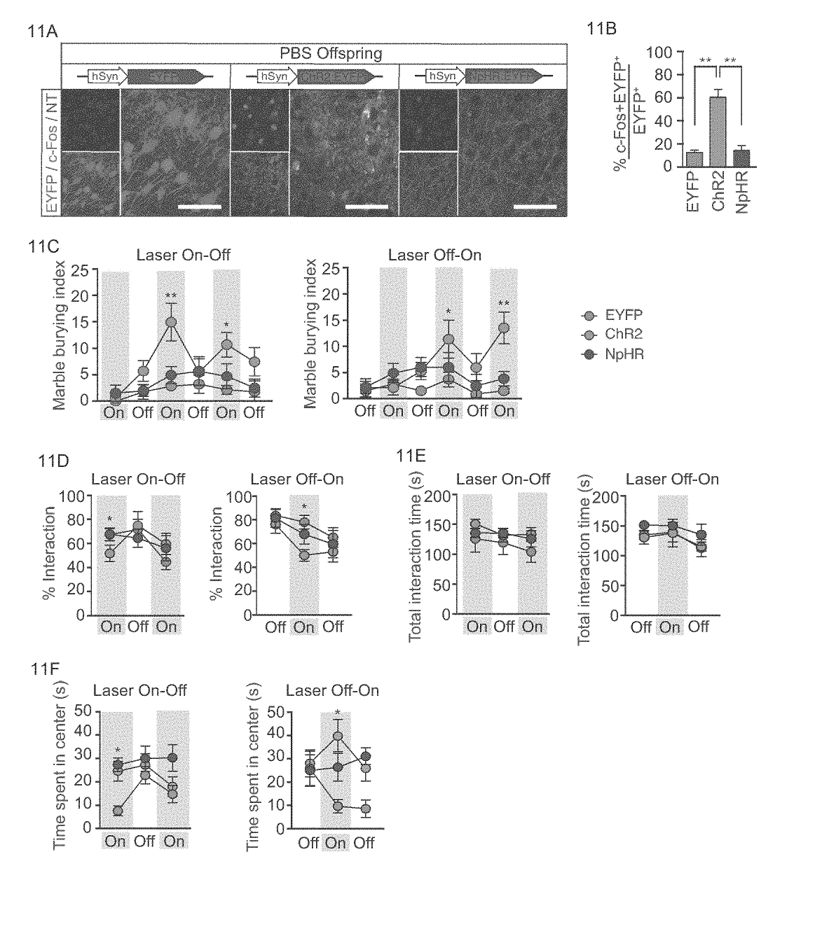

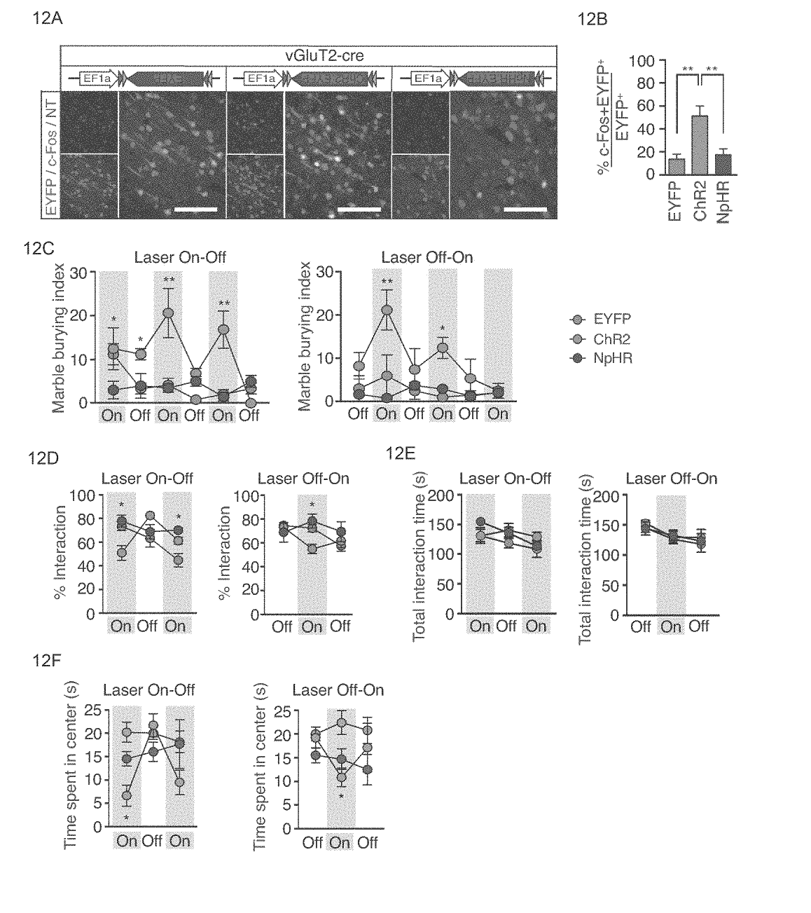

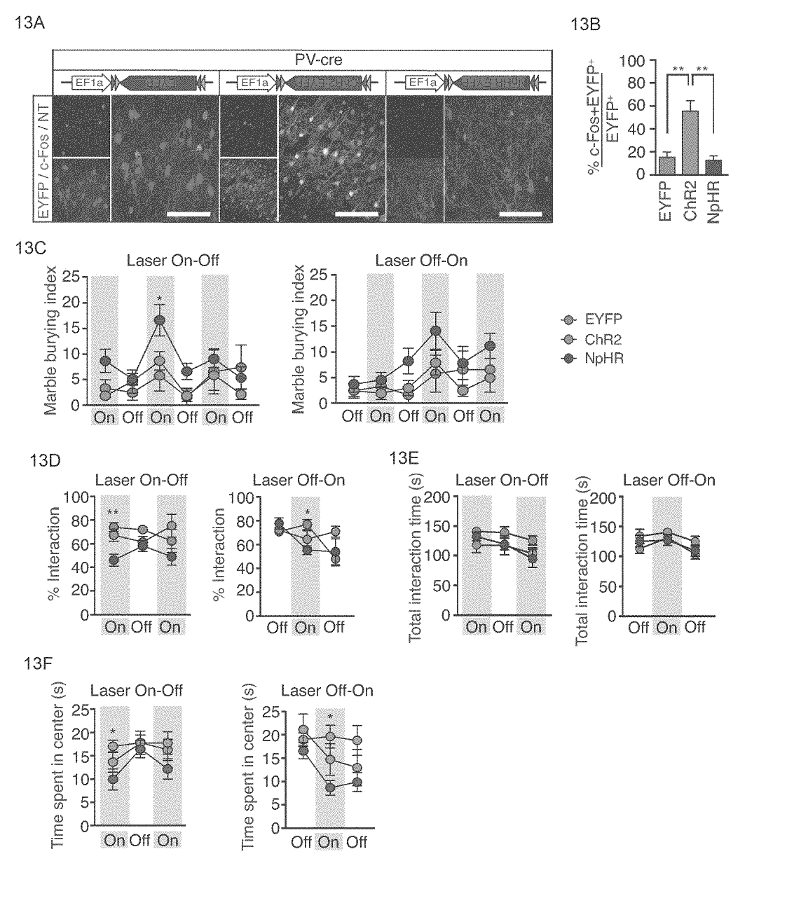

The characterization of cortical patches indicated that an increase in the overall neural activity within S1 could be a major factor driving abnormal behavioral phenotypes in MIA offspring (FIG. 1f, g and FIG. 8). Indeed, previous studies suggested that deficits in interneuron development and subsequent perturbations of excitation/inhibition (E/I) balance could be the underlying cause(s) of ASD²²⁻²⁸. To explore this issue further, the present inventors asked if increasing neural activity in S1 could recapitulate MIA-induced behaviors in WT adult animals. The present inventors virally expressed either Enhanced Yellow Fluorescent Protein (EYFP), channelrhodopsin (ChR2)²⁹, or halorhodopsin (NpHR)³⁰ using the neuronal specific promoter, human Synapsin 1 (hSyn1) (FIG. 3c and FIG. 11A). Both the virus and the optical fibers were bilaterally targeted to a region centered on S1DZ (S1DZ region), where cortical patches were most consistently observed in MIA animals (FIG. 3a). Animals were subsequently subjected to the behavioral assays described above, while delivering optical stimulation at 3-minutes intervals (a 3 minute-‘On’ session followed by a 3 minute-‘Off’ session or vice versa) (FIG. 3b). Increasing neural activity with ChR2 in WT offspring resulted in enhanced marble burying behaviors, impaired sociability without any effects on total interaction time, and reduced time spent at the center of an open field (FIG. 3d-g and FIG. 11). On the other hand, photostimulation of EYFP-expressing neurons in the control group failed to induce any MIA-associated behaviors. Furthermore, reducing neural activity in S1 using NpHR did not generate behavioral abnormalities, with the exception of a slight, yet significant, increase in marble burying behavior compared to the EYFP-expressing control group (FIG. 3d). This effect is likely due to non-specific inhibition of different types of neurons in the photostimulated region. Therefore, to specifically isolate the contribution of only excitatory glutamatergic neurons, a Cre-dependent strategy was used to express opsins under the control of the vesicular Glutamate Transporter 2 promoter (vGluT2) (FIG. 3h)³¹. Increasing activity of vGluT2⁺ neurons using ChR2 recapitulated all three MIA-associated behaviors, while photostimulation in NpHR- or EYFP-expressing animals failed to induce these behavioral abnormalities (FIG. 3i-1, FIG. 12). The present inventors also selectively modulated neural activity in PV⁺ neurons by virally driving Cre-dependent opsin expression in PV-Cre animals (FIG. 3m)³². Inhibiting the activity of the PV⁺ neuronal population with NpHR mimicked the loss of PV⁺ neurons observed in the MIA-cortical patches and recapitulated all three MIA-associated behavioral phenotypes (FIG. 3n-q, FIG. 13). On the other hand, photostimulation of EYFP- or ChR2-expressing animals did not produce any observable deficits (FIG. 3n-q).

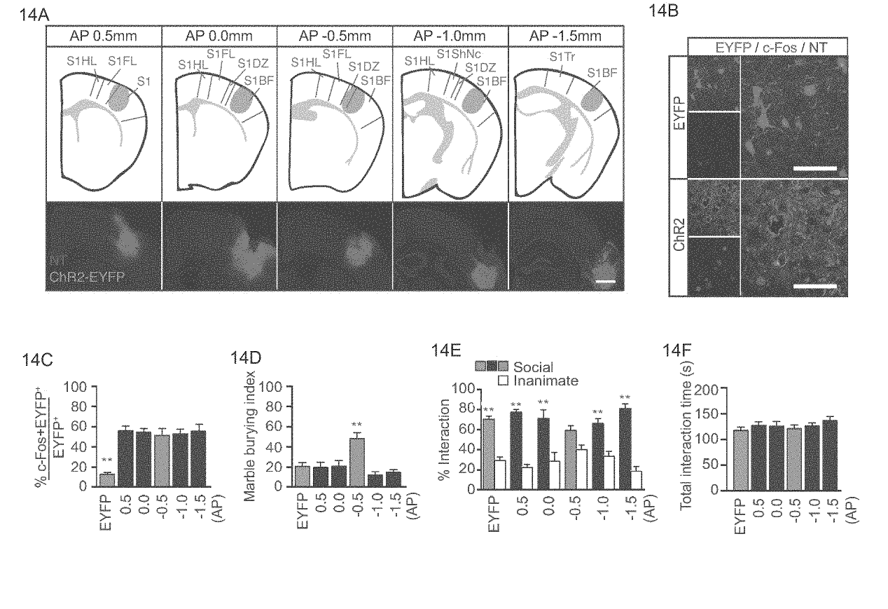

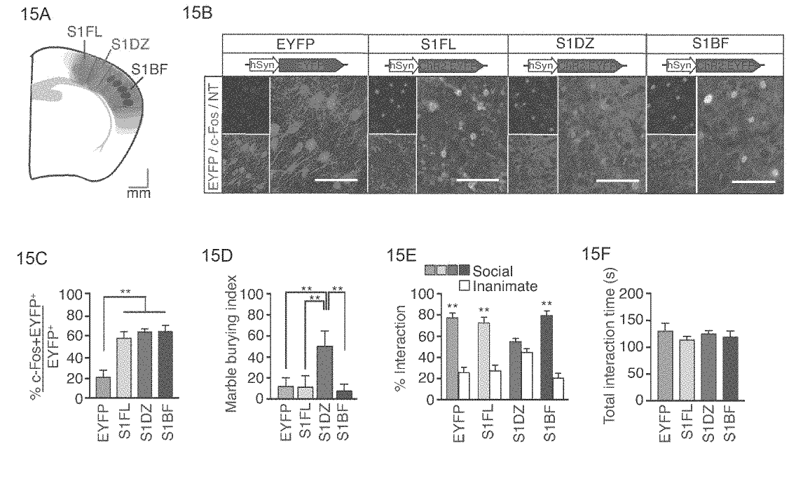

The present inventors next examined whether the ability to drive these MIA behaviors is a general feature of S1 or is specific to the S1DZ region (˜AP=−0.5), where cortical patches were predominantly observed. ChR2 and optical implants were targeted into four additional anterior and posterior regions of S1, while keeping the ML coordinates consistent (FIG. 14A-C). Photostimulation of these off-target regions did not elicit MIA phenotypes in the marble burying or sociability assays (FIG. 14D-F). The same manipulation carried out medially in the forelimb region (S1FL) or laterally in the barrel field of primary somatosensory cortex (S1BF) also failed to induce any behavioral abnormalities (FIG. 15). These results demonstrate that MIA-like behaviors can be recapitulated in WT animals either through the activation of excitatory neurons or the inhibition of PV⁺ inhibitory neurons and that this feature is primarily localized to the cortical region centered on the S1DZ.

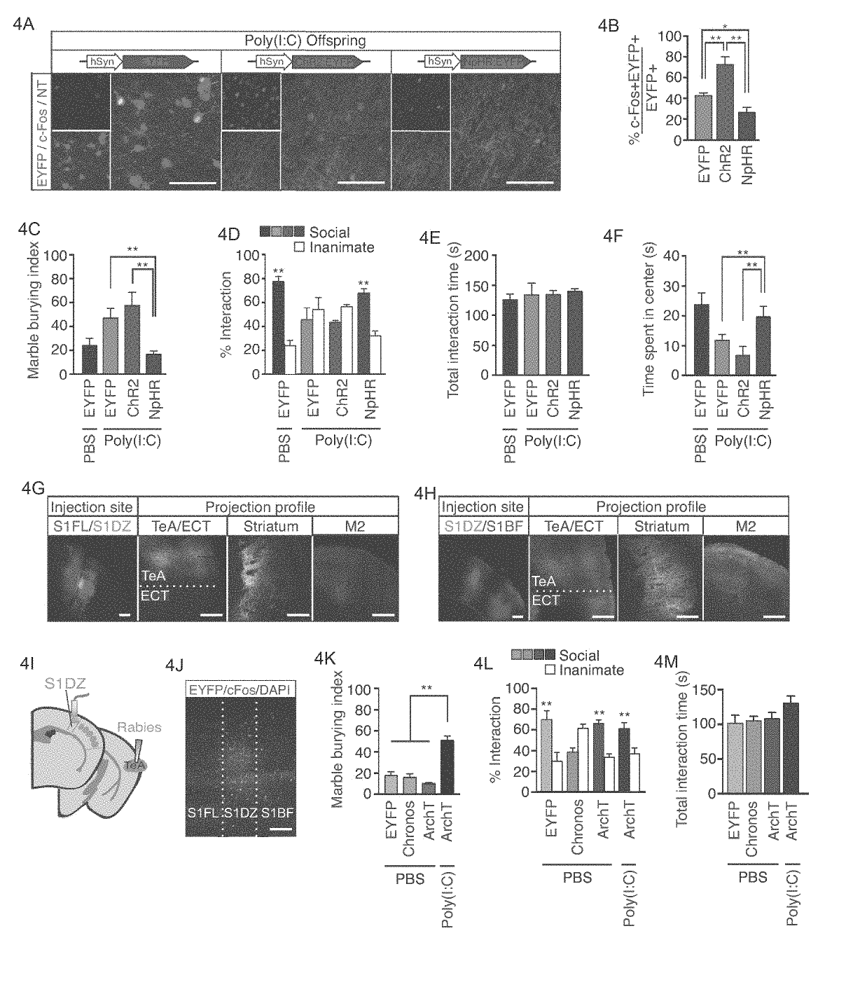

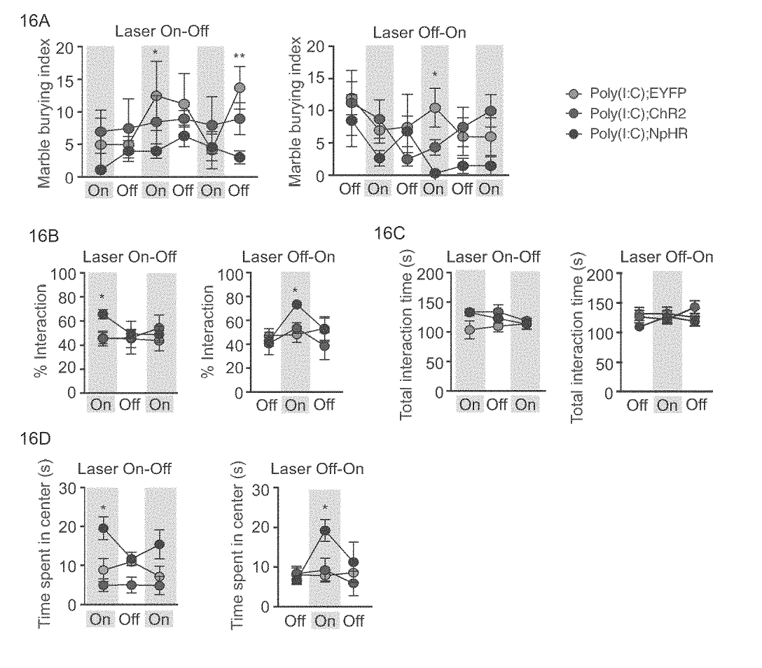

The present inventors next asked whether reduction of neural activity in the S1DZ region of MIA offspring is sufficient to correct the observed behavioral abnormalities. Photostimulation of NpHR-expressing animals decreased the number of c-Fos⁺ cortical neurons when compared to those in photostimulated EYFP-expressing MIA animals (FIG. 4a,b). This inhibition of neural activity was sufficient to suppress enhanced marble burying, to restore sociability, and to increase the time spent in the center of the open field to levels observed in control PBS animals (FIG. 4c-f and FIG. 16). Photostimulation of EYFP- or ChR2-expressing animals did not rescue any behavioral deficits (FIG. 4c-f). Thus, the foregoing data demonstrate that acute reduction in neural activity in the S1DZ region is sufficient to rescue ASD-like behavioral phenotypes in MIA offspring prenatally exposed to maternal inflammation.

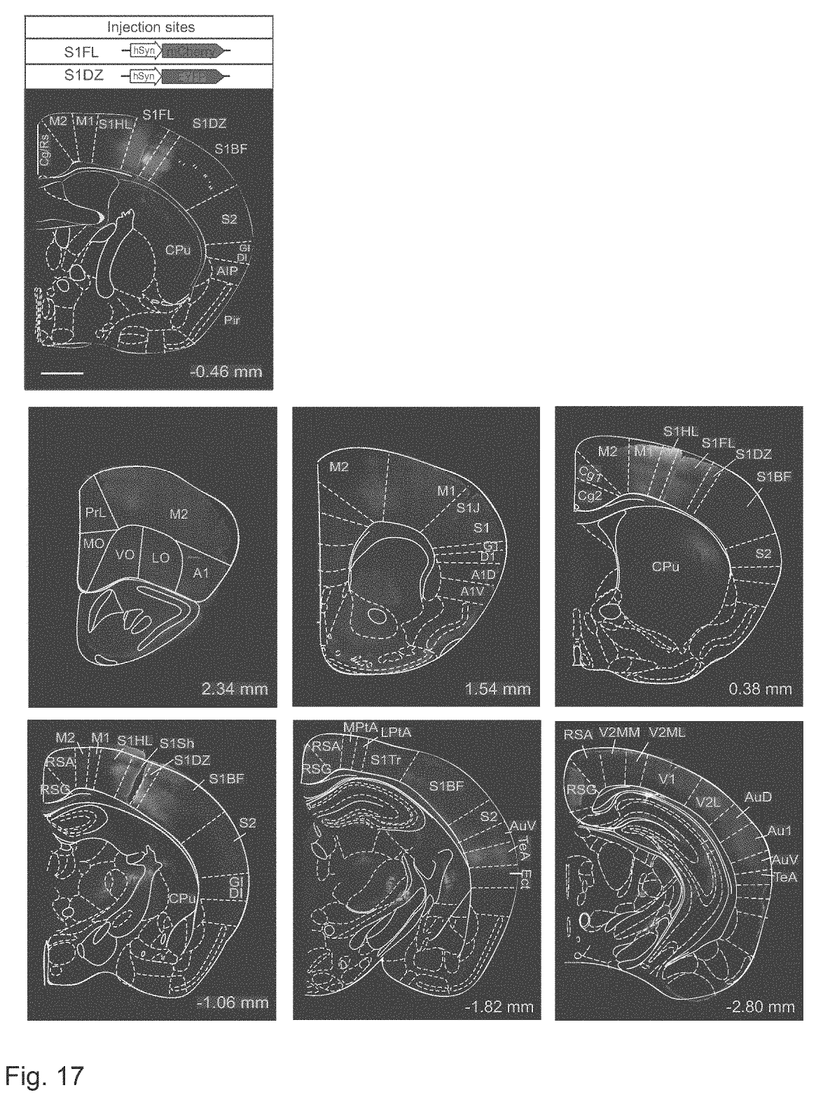

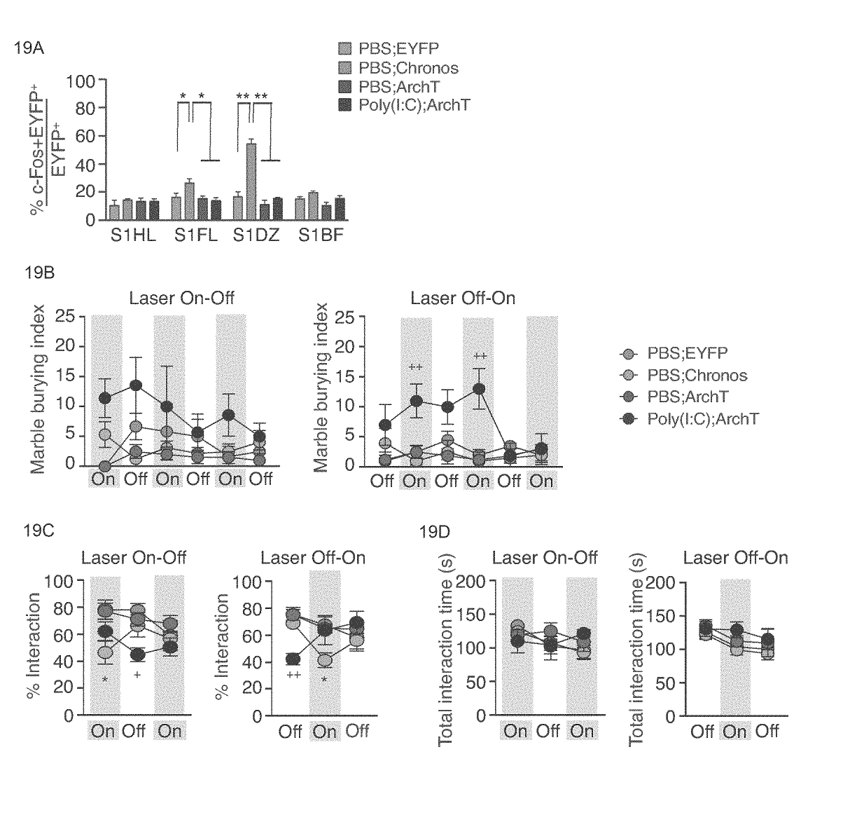

To gain insight into the downstream neural circuits involved in eliciting MIA-associated behaviors, the efferent targets of the S1DZ were next examined. The present inventors injected an anterogradely-labeling adeno-associated virus (AAV) driving EYFP into the S1DZ and an AAV driving mCherry into either the S1FL or S1BF (FIG. 4g, h and FIG. 17, 18). These tracing studies revealed that the S1DZ exhibits largely distinct efferent targets compared to the S1FL and S1BF regions (FIG. 4g, h and FIG. 17, 18). The S1DZ selectively sends axons to a sub-region of M2 and the striatum as well as the TeA (FIG. 4g, h and FIG. 17, 18). To test the role of these distinct downstream regions in eliciting MIA-behaviors, the present inventors injected retrogradely transported rabies virus (RV)³³ expressing EYFP, Chronos (excitatory opsin)³³, or ArchT (inhibitory opsin)^{34,35} into the TeA to retrogradely label S1DZ neurons in WT animals (FIG. 4i, j). Photostimulation of Chronos-positive neurons in the S1DZ generated sociability deficits without affecting total interaction time, but failed to induce increased marble burying phenotypes (FIG. 4k-m and FIG. 19). Photostimulation of EYFP- or ArchT-expressing neurons did not produce any behavioral deficits (FIG. 4k-m and FIG. 19). Thus, in otherwise WT animals, increasing the neural activity of the neurons from the S1DZ that project to the TeA recapitulated MIA-associated sociability deficits, but not the repetitive marble burying phenotypes. Conversely, in MIA animals, decreasing the neural activity of this S1DZ neuronal population with ArchT restored normal sociability, but failed to suppress the enhanced marble burying phenotype (FIG. 4k-m and FIG. 19). These data suggest that the neural connection from the S1DZ to the TeA selectively modulates interactions with social targets without impacting repetitive behaviors.

Identifying neural circuits and components that modulate behaviors aberrantly manifested in ASD patients is critical for developing therapeutic approaches. One challenge to achieving this goal is the paucity of animal models, in which discrete brain areas are known to mediate autism-like behaviors. Many rodent models that are developed to study genetic contributions to the etiology of ASD are often found with structural and functional abnormalities throughout the brain, making it difficult, if not impossible, to systematically identify neural substrates that functionally drive behavioral deficits. Here the present inventors identified a restricted brain region centered on S1DZ and its efferent target TeA as components of the neural circuit that mediates ASD-like behavioral abnormalities. The somatosensory pathway has been suggested as a potential neural substrate that mediates behavioral abnormalities in various mouse models of ASD¹⁷⁻¹⁹. Furthermore, it has been suggested that ASD patients more heavily depend on proprioceptive inputs and they have difficulty integrating proprioceptive information with inputs from other senses³⁶⁻³⁹. The observation that the neurons in the S1DZ projecting to the TeA affect sociability, but not marble burying behavior, suggests that the S1DZ may process afferent information via segregated neural pathways to influence distinct aspects of ASD-like behaviors. Furthermore, it is interesting to note that the main efferent targets of S1DZ, such as the TeA and M2, are other locations in which cortical patches are frequently found in MIA offspring (FIG. 1b and FIG. 6). This observation suggests that cortical patch formation in MIA offspring may be intricately linked to concerted neural activity among the connected brain regions. It also should be noted that in some MIA animals, cortical patches were only observed outside of S1 (12% of animals, n=50), suggesting that S1DZ might be one of the nodes within a larger network that controls MIA-impacted behaviors. The MIA mouse model with discrete, functionally-relevant perturbations in S1DZ provides access to the network that may modulate affected behaviors across different mouse models of autism.

Given that developing new and efficacious therapeutic regimens for treating ASD in humans remains an unmet need, the present inventors sought to explore cortical developmental abnormalities that contribute to and/or are causally related to ASD and MIA-induced cortical developmental abnormalities and behavior in offspring of pregnant dams in whom MIA has been induced so as to devise therapeutic regimens that target these cortical abnormalities in affected offspring.

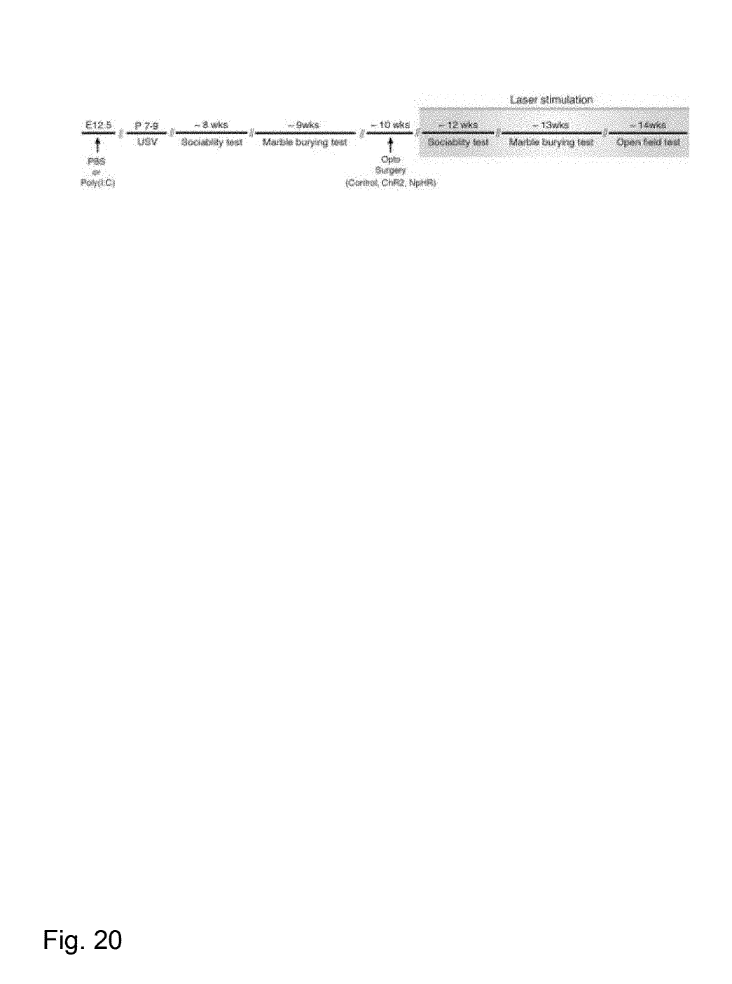

A schematic depicting a timeline for a particular embodiment directed to treating such offspring is presented in FIG. 20. The particular embodiment shown relates to surgical intervention combined with introduction of optogenetic tools. It will be appreciated that a pharmacological agent or combination thereof or chemogenetic tools could be introduced via similar or different means so as to achieve delivery of the therapeutic intervention to the cortex, S1DZ, and/or abnormal cortical patch of a subject in need thereof.

The present inventors also envison treating subjects who exhibit ASD or ASD-like phenotypes, but for whom little or no information is available regarding potential for in utero exposure to MIA, either with pharmacological agents (such as GABAergic receptor agonists) or with optogenetic or chemogenetic tools (or other non-invasive neural activity modulating techniques) to correct dysregulated neuronal E/I ratio.

The aforementioned pharmacological agonists and/or optogenetic or chemogenetic tools may be administered or used alone or in conjunction with agents that modulate IL-17 activity, including: agents that promote IL-17 activity, including exogenous IL-17a and/or IL-17f (e.g., synthetic/manmade IL-17a or IL-17f); or inhibitors of IL-17 activity (e.g., antagonistic IL-17 antibodies) and/or inhibitors of IL-17 or RORγt activity. Agents that modulate IL-17 activity may also be used alone to treat subjects who exhibit ASD or ASD-like phenotypes irrespective of whether or not there is any information available pertaining to potential exposure of such subjects to MIA in utero.

It will be understood that subjects who exhibit ASD-like phenotypes, including those who are offspring of mothers identified as having elevated levels of IL-17a in their sera while carrying the subject in utero, can be treated as described herein at any age. Exemplary such ages include, infants, children, adolescents, and adults with respect to human subjects, and equivalent stages with respect to other mammalian subjects, such as mice, rats, and primates.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

In a broad aspect, methods are disclosed herein for treating a subject afflicted with a psychiatric disorder [e.g., autism spectrum disorder (ASD), schizophrenia, and/or depression], wherein such methods call for selecting a subject afflicted with the psychiatric disorder who was exposed in utero to maternal immune activation (MIA) or in whom dysregulated E/I ratios are detected or visualized and treating the subject by administering pharmacological agents (such as GABAergic receptor agonists or modulators of IL-17activity) or implementing optogenetic or chemogenetic tools to correct dysregulated neuronal excitation/inhibition (E/I) ratios in the cortex of the subject. In a particular embodiment thereof, the pharmacological agents and/or optogenetic or chemogenetic tools are targeted specifically to patches of cortical malformation in the subject.

### Optogenetic Tools

The opsin genes encode a family of seven-transmembrane, light-responsive proteins. Opsin genes comprise two distinct superfamilies: microbial opsins (type I) and animal opsins (type II). Of the microbial opsins, channelrhodopsins, halorhodopsins, and OptoXRs are most commonly used as optogenetic tools. Channelrhodopsins conduct cations and depolarize neurons upon illumination; halorhodopsins (e.g., NpHR, which is an endoplasmic reticulum trafficking-enhanced version of halorhodopsin) conduct chloride ions into the cytoplasm upon yellow light illumination; and OptoXRs are rhodopsin-GPCR (G protein—coupled receptor) chimeras that respond to green (500 nm) light with activation of the biological functions dictated by the intracellular loops used in the hybrid. See, for example, Fenno et al. (2011, Annual Rev Neurosci 34:389-412) and Dugue et al. (2012, Progress in Brain Research 196:1-28), the entire content of each of which is incorporated herein by reference.

Specific targeting of optogenetic tools may be achieved by use of viral expression systems, such as those described in Fenno et al. (2011, Annual Rev Neurosci 34:389-412) and Dugue et al. (2012, Progress in Brain Research 196:1-28). As described therein, viral expression systems can be designed to possess cell specificity by virtue of specific promoters and also by spatial targeting of the virus injection and by restriction of opsin activation to particular cells or projections thereof via targeted light delivery. Such means are described in, for example, Zhang et al. (2010, Nat Protoc 5:439-456) and Diester et al. (2011, Nat Neurosci 14:387-397), the entire content of each of which is incorporated herein by reference. Stimulation in vivo is typically achieved with laser light delivered to the transduced tissue via optical fibers inserted through implanted cannulas or with fiber-coupled high-power LEDs. Chronic delivery of light using implanted infrared-triggered LEDs is also being developed and shows promise. See, for example, Fenno et al. (2011, Annual Rev Neurosci 34:389-412) and references cited therein, which are incorporated herein by reference in their entireties. Readouts from optogenetically controlled tissue can be obtained using optrodes or silicon multisite electrodes and movable tetrode arrays combined with optical fibers. See, for example, Fenno et al. (2011, Annual Rev Neurosci 34:389-412) and references cited therein, which are incorporated herein by reference in their entireties.

Optogenetics has, moreover, also been implemented to assess potential therapeutic mechanisms underlying cortical intervention in mouse models of depression (Covington et al. (2010, J Neurosci 30:16082-16090) and to study amygdala circuits involved in fear and anxiety (Llewellyn et al. (2010, Nat Med 16:1161-1165). See also Reardon (2016, Nature doi:10.1038/nature.2016.19886; the entire content of which is incorporated herein by reference along with the references cited therein), which presents a review of advances in the field of optogenetics and implementation thereof in clinical trials in humans

Additional references describing optogenetics and implementation thereof are as follows: Deisseroth (2015, Nat Neuro 18:1213), Steinberg et al. (2015, Curr Opin Neurobiol 30:9), and Warden et al. (2014, Ann Rev Biomed Eng 16:103), the entire content of each of which is incorporated herein by reference.

### Chemogenetic Tools

Chemogenetic tools, including genetically engineered receptors that can be targeted to specific cell types and are engineered to interact selectively with small molecules, are also envisioned as having utility in methods described herein. Such chemogenetic tools are described in, for example, Sternson et al. (2014, Annu Rev Neurosci 37:387) and Urban et al. (2014, Ann Rev Pharmacol Toxicol 55:399), the entire content of each of which is incorporated herein by reference.

### Visualization of Cortical Patches

Non-invasive detection methods are available for visualizing abnormal cortical patches. Functional magnetic resonance imaging (fMRI) can, for example, be used to map regions of cortical activity and dysfunction. See, for example, Dupont (2008, Epilepsies 20:250), the entire content of which is incorporated herein by reference. Additional neuroimaging techniques such as electroencephalography (EEG), electroencephalography-fMRI (EEG-fMRI), fluorodeoxyglucose positron emission tomography (FDG PET), magnetoencephalography (MEG), diffusion tensor imaging (DTI), and intra-cranial EEG are known and have been described in, for example, Thornton et al. (2011, Ann Neurol 70:822) and Kabat et al. (2012, Pol J Radiol 77:35), the entire content of each of which is incorporated herein by reference. Several of the aforementioned techniques have, moreover, been applied to evaluating autistic subjects as described in, for example, Valvo et al. (2016, Eur Child Adolesc Psychiatry 25:421-429) and Boutros et al. (2015, Neuropsychiatric Electrophysiology 1:3), the entire content of each of which is incorporated herein by reference.

### Administration

As described herein, pharmacological agents and/or optogenetic and/or chemogenetic tools may be administered or used alone or in conjunction with agents that modulate IL-17 activity, including: agents that promote IL-17 activity, including exogenous IL-17a and/or IL-17f (e.g., synthetic/manmade IL-17a or IL-17f); or inhibitors of IL-17 activity (e.g., antagonistic IL-17 antibodies) and/or inhibitors of IL-17, IL-17R (antagonists of IL-17R), or RORγt activity. Agents that promote IL-17 activity also include agonists of the IL-17R, including IL-17 mimics and IL-17R antibodies that bind to and promote IL-17R activity and downstream signaling. Such pharmacological agents and/or optogenetic and/or chemogenetic tools may be administered systemically or directly into the cerebrospinal fluid (via, e.g, intrathecal injection) or delivered to the cortex, S1DZ, and/or patches of cortical malformation, such as those described herein.

As described herein, agents that modulate IL-17 activity may be administered as the sole therapeutic agents and may, moreover, be administered systemically or directly into the cerebrospinal fluid (via, e.g, intrathecal injection) or delivered to the cortex, S1DZ, and/or patches of cortical malformation, such as those described herein.

The use of one or more agents or compounds or optogenetic or chemogenetic tools that correct dysregulated neuronal E/I ratios in the cortex for treating a subject afflicted with the psychiatric disorder is also encompassed herein as is its/their use in the preparation of a medicament for treating the psychiatric disorder in a subject.

### GABA Signaling Pathways

GABA is the major inhibitory neurotransmitter in the adult brain. It is, moreover, critical for normal development and regulation of neurotransmission. In the adult, GABA inhibits neuronal firing by activating two major families of receptors expressed in the mammalian brain: GABA_{A} receptors, which are ligand-gated ion channels that promote chloride fluxes, and GABA_{B} receptors, which are G-protein coupled receptors. In adults, GABA_{A} receptor activation promotes chloride influx and hyperpolarization of the membrane, decreasing neuronal excitability. During fetal development, however, chloride gradients across the membrane are reversed, so activation of GABA_{A} receptors in the hippocampus and neocortex causes net chloride efflux and enhanced excitation (Cherubini et al. 1991, Trends Neuroscience 14: 515-519).

### GABAergic Receptor Agonists

Agents capable of positive allosteric modulation of GABAergic transmission include, without limitation, GABA (e.g., synthetic/manmade GABA), muscimol, barbiturates (e.g., phenobarbital), benzodiazapines, diazepam, triazolam, alprazolam, clonazepam, the benzoquinolizinone Ro 19-8022 [(R)-1-[(10-chloro-4-oxo-3-phenyl-4H-benzo[a]quinolizin-1-yl)carbonyl]-2-pyrrolidine-methanol], bretazenil, β-carboline abecarnil (isopropyl-6-benzyloxy-4-methoxymethyl-b-carboline-3-carboxylate), abecarnil, cyclopyrrolone pagoclone, imidazenil and SL651498 [6-fluoro-9-methyl-2-phenyl-4-(pyrolidin-1-yl-carbonyl)-2,9-dihydro-1H-pyrido[3,4-b]indol-1-one], zopiclone, ocinaplon (pyrazolo[1,5-a]-pyrimidine), the pyridoindole SL651498, and L-838,417. These compounds are known in the art and described in, for example, Farb et al. (2014, Pharmacological Reviews 66:1002-1032) and Cellot et al. (2014, Frontiers in Pediatrics 2:Article 70), the entire content of each of which is incorporated herein by reference. See also the National Institutes for Health website pertaining to Clinical Trials NCT01966679, the entire content of which is incorporated herein in its entirety.

Agents that increase synaptic concentration of GABA, either by inhibiting uptake or degradation, may also be useful for increasing GABAergic neurotransmission. Such agents include, without limitation, GABA-transaminase inhibitors, such as vigabatrin (γ-vinyl-GABA) and valproic acid (VPA). These compounds are known in the art and described in, for example, Farb et al. (2014, Pharmacological Reviews 66:1002-1032).

Agents that reverse the polarity of the GABA responses from the depolarizing to the hyperpolarizing direction may also be useful for increasing GABAergic neurotransmission. Use of the diuretic bumetanide, a selective blocker of the cation-chloride importer NKCC1, is envisioned as an exemplary agent having this functionality.

### Modulators of IL-17 Activity

Exemplary compounds for the methods and uses described herein include agents or compounds that modulate IL-17 activity, including: exogenous IL-17a and/or IL-17f (e.g., synthetic/manmade/recombinant IL-17a or IL-17f), IL-17 mimics, and IL-17R agonists to promote IL-17 activity and antagonistic IL-17 antibodies and/or inhibitors of IL-17 or IL-17R activity and/or inhibitors of RORγt activity (e.g., small molecule inhibitors).

Exemplary inhibitors of RORγt activity include: TMP778 (Skepner et al. 2014, J Immunol 192:2564-2575), SR1001 (Solt et al. 2011, Nature 472:491), SR1555 (Solt et al. 2012, ACS Chem Biol 7:1515), and SR2211 (Kumar et al. 2012, ACS Chem Biol 7:672). These and other inhibitors of RORγt activity, as well as assays for detecting/assessing RORγt activity, are described in, for example, U.S Patent Application Publication 2013/0085162, 2013/0065842 and 2007/0154487; U.S. Pat. No. 9,101,600; WO2013/036912, WO2012/074547, WO2013/079223, WO2013/178362, WO2011/112263 (SR-9805), WO2011/112264, WO2010/049144, WO2012/027965, WO2012/028100, WO2012/100732, WO2012/100734, WO2011/107248, WO2012/139775, WO2012/064744, WO2012/106995, WO2012/147916, and WO2010/049144, the entire content of each of which is incorporated herein by reference. Other inhibitors of RORγt activity are also described in Skepner et al. (2014, J Immunol 192:2564-2575), Skepner et al. (2015, Immunology doi: 10.111/imm.12444, epub ahead of print), Nishiyama et al. (2014, Bioorganic & Medicinal Chemistry 22:2799-2808; compound 5b), Fauber et al. (2014, J Medicinal Chem 57:5871-5892), Mele et al. (2013, J Exp Med 210:2181-2190), Dhar et al. (2013, Annual Reports in Medicinal Chemistry 48:169-182), Xu et al. (2011, J Biol Chem 286:22707; ursolic acid), and Huh et al. (2011, Nature 472:486-490), the entire content of each of which is incorporated herein by reference. With respect to Fauber et al. and Dhar et al., in particular, each of the references cited therein is also incorporated herein by reference in its entirety.

Exemplary inhibitors of IL-17 activity include antibodies specific for IL-17a or the IL-17R that antagonize the activity of either of IL-17a or IL-17R. In a particular embodiment, the inhibitor of IL-17 activity is a human monoclonal antibody or a humanized monoclonal antibody. Such antibodies are envisioned as being able to block IL-17R engagement by IL-17A. In a more particular embodiment, the human monoclonal antibody is brodalumab (AMG 827), which is specific for the IL-17R. In another particular embodiment, the humanized monoclonal antibody is ixekizumab (LY2439821) or secukinumab (AIN457), which are specific for IL-17A. Also envisioned for use in methods described herein are antibodies specific for the p19 subunit of IL-23 or the p40 subunit of IL-23 and IL-12. Exemplary antibodies specific for the p19 subunit of IL-23 include MK-3222 (SCH 900222), CNTO 1959, and AMG 139. Exemplary antibodies specific for the p40 subunit of IL-23 and IL-12 include Stelara (ustekinumab; CNTO 1275).

Specific agents/compounds from each of the categories listed above are available commercially as follows: brodalumab (AMG 827) and AMG 139 are available from Amgen/Medlmmune; ixekizumab (LY2439821) is available from Eli Lilly; secukinumab (AIN457) is available from Novartis; MK-3222 (SCH 900222) is available from Merck; CNTO 1959 and Stelara (ustekinumab; CNTO 1275) are available from Janssen Biotech (J & J).

Also envisioned for use in methods described herein are antibodies for other Th17 cell specific cytokines, such as, but not limited to IL-17f and IL-22. Antibodies and reagents specific for Th17 specific cell surface proteins, of which CCR6 is an example, are also envisioned for use in methods described herein. See also Hedrick et al. (2010, Expert Opin Ther Targets 14:911-922), the entire content of which is incorporated herein by reference). Blocking antibodies specific for IL-23 receptor are also envisoned for use in methods described herein.

Also envisioned for use in methods described herein are antibodies for the IL-23 receptor (IL-23R). See, for example, US 2014/0275490, which is incorporated herein in its entirety by reference.

Also envisioned herein are antibody fragments or altered/mutated antibodies, particularly those wherein the Fc domain is absent or altered/mutated such that the antibody fragment or mutated antibody can no longer bind to Fc receptors. Methods for generating antibody fragments or mutated antibodies that can no longer bind to Fc receptors are described in Firan et al. (2001, Intern Immunol 13:993-1002), the entire content of which is incorporated herein by reference.

### Modulators of IFN-γ Activity

Exemplary compounds for the methods and uses described herein include agents or compounds that modulate IFN-γ activity, including: exogenous IFN-γ (e.g., synthetic/manmade/recombinant IFN-γ), IFN-γ mimics (including peptidomimetics, and IFN-γR agonists to promote IFN-γ activity and antagonistic IFN-γ antibodies and/or inhibitors of IFN-γ or IFN-γR activity (including, e.g., small molecule inhibitors). See, for example, Filiano et al. (2016, Nature 535:425-429), Sundberg et al. (2014, Curr Opin Chemical Biology 23:23-30), Mujtaba et al. (2006, Clin Vaccine Immunol 13:944-952) and Huxley et al. (2004, Chemistry & Biology 11:1651-1658); the entire content of each of which is incorporated herein by reference. Recombinant IFN-γ and mimics thereof are also commercially available from a number of commercial suppliers including:R&D Systems, ThermoFisher Scientific, Abcam, Sigma-Aldrich, and InvivoGen.

One skilled in the art can readily determine or assess the suitability of other compounds for use in the invention by screening in cellular assays of IL-17 activity or IFN-γ activity such as those described herein or known in the art, or in animal models of disease in which IL-17 and/or IFN-γ cell activity is implicated such as those described herein and elsewhere. With regard to IL-17, see, for example, U.S Patent Application Publication No. 2007/0154487, the entire content of which is incorporated herein by reference.

Further to the above, an MIA rhesus monkey model has also been described. See, for example, Bauman et al. (2014, Biol Psychiatry 75:332-341), which is incorporated herein by reference in its entirety. In the rhesus monkey model, pregnant rhesus monkeys in whom MIA has been induced give birth to offspring with abnormal repetitive behaviors, communication, and social interactions. Accordingly, both the MIA mouse model and the MIA rhesus monkey model provide suitable animal models in which to examine the effects of an activated maternal immune system on fetal development and the ramifications thereof in the offspring subsequently born. These animal models also provide suitable in vivo assays for evaluating potential therapeutics for the treatment of offspring who are at risk for developing autism and schizophrenia or display symptoms of autism or schizophrenia.

Therefore, if appearing herein, the following terms shall have the definitions set out below.

The term “hyperinflammatory condition” as used herein refers to a condition in a subject wherein Th17 cell activity and potentially that of related T cells, such as, for example, CD8 and γδT cell receptor (TCR) T cells with similar RORγt-dependent cytokine programs is elevated relative to a suitable control subject. With regard to pregnant females, it is understood that Th17 cell activity is elevated relative to non-pregnant females. Accordingly, a “hyperinflammatory condition” in a pregnant female induced, for example, by environmental conditions, toxins, and/or an infection (e.g., viral, bacterial, or fungal) is compared relative to that of a pregnant female that has not been exposed to the aforementioned inducers or the like.

The term “correct” or “improve” as used herein with respect to dysregulated neuronal excitation/inhibition (E/I) ratios in the cortex of a subject refers to at least partially restoring a normal E/I ratio that is comparable to or similar to that of a wildtype or normal E/I ratio. Accordingly, “correct” or “improve” relate to altering or modifying the dysregulated neuronal E/I ratios in the cortex of a subject.

“Preventing” or “prevention” refers to a decreased likelihood of acquiring a disease or disorder. The term may be used to encompass a decreased likelihood of at least 50%, at least 60%, at least 70%, at least 80%, at least 90%, at least 95%, at least 96%, at least 97%, at least 98%, or at least 99% relative to a control subject.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means the amount of a compound that, when administered to a subject for treating a disease or disorder, is sufficient to effect such treatment for the disease or disorder. The “therapeutically effective amount” can vary depending on the compound, the disease and its severity, and the age, weight, etc., of the subject to be treated.

The term ‘treating’ or ‘treatment’ of any disease or infection refers, in one embodiment, to ameliorating the disease or infection (i.e., arresting the disease or growth of the infectious agent or reducing the manifestation, extent or severity of at least one of the clinical symptoms thereof). In another embodiment ‘treating’ or ‘treatment’ refers to ameliorating at least one physical parameter of the disease. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

The agents and compounds and derivatives thereof of use in the invention may be prepared in pharmaceutical compositions, with a suitable carrier and at a strength effective for administration by various means to a subject in need thereof, such as a subject exhibiting ASD-like phenotype. A variety of administrative techniques may be utilized, among them parenteral techniques such as subcutaneous, intravenous and intraperitoneal injections, catheterizations and the like. Average quantities of the agents and compounds and derivatives thereof may vary and in particular should be based upon the recommendations and prescription of a qualified physician or veterinarian.

The present invention further contemplates therapeutic compositions useful in practicing the therapeutic methods of this invention. A subject therapeutic composition includes, in admixture, a pharmaceutically acceptable excipient (carrier) and one or more of an agent, compound or derivative thereof, as described herein as an active ingredient.

The preparation of therapeutic compositions which contain agents, compounds, or derivatives thereof as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

An agent, compound, or derivative thereof can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic agent, compound, or derivative thereof-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or cell modulation desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and subsequent shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Methods and Materials

Animals:

All experiments were performed in accordance with the Guide for the Care and Use of Laboratory Animals and were approved by the National Institutes of Health and the Committee and Animal Care at Massachusetts Institute of Technology. All C57BL/6 mice were purchased from Taconic (USA) and Nestin-cre (003771), PV-cre (008069), and vGluT2-cre (016963) mice from Jackson laboratory (USA). IL-17Ra^{fl/flf} mice were described previously⁴⁰. All mice were crossed and maintained in-house with C57BL/6 mice from Taconic. IL-17Ra″ animals were crossed with Nestin-cre to remove IL-17Ra in the brain. The following primers were used to genotype progenies: IL-17Ra-flox-1-F 5′-GGCAGCCTTTGGGATCCCAAC-3′, IL-17Ra-flox-2-R 5′-CTACTCTTCTCACCAGCGCGC-3′ for WT 336 bps/Floxed 377 bps; IL-17Ra-flox-2-R, IL-17Ra-flox-3-F 5′-GTGCCCACAGAGTGTCTTCTGT-3′ for KO 478 bps; and Cre-F 5′-GCGGTCTGGCAGTAAAAACTATC-3′, Cre-R 5′-GTGAAACAGCATTGCTGTCACTT-3′ for Nestin-cre 100 bps.

Maternal Immune Activation:

Mice were mated overnight and females were checked daily for the presence of seminal plugs, noted as embryonic day 0.5 (E0.5). On E12.5, pregnant female mice were weighed and injected with a single dose (20 mg/kg i.p.) of poly(I:C) (Sigma Aldrich, USA) or PBS vehicle. Each dam was returned to its cage and left undisturbed until the birth of its litter. All pups remained with the mother until weaning on postnatal day 21 (P21), at which time mice were group housed at a maximum of 5 per cage with same-sex littermates. For checking the effect of poly(I:C) administration at different time points during pregnancy, the present inventors injected poly(I:C) into pregnant dams on E12.5, E15.5, or E18.5.

Stereotaxic Injection:

All surgeries were carried out using aseptic techniques and animals were pre-operatively injected with the following dosages of anesthetics and analgesics: ketamine (100 mg/kg i.p.), xylazine (10 mg/kg i.p.), and slow-release buprenorphine (1 mg/kg s.c.). All stereotaxic reference points were set at Bregma for the AP axis, at the midline for the ML axis, and at the surface of the brain for the DV axis. For optogenetic experiments, animals received bilateral stereotaxic injections of one of the following viruses at rates of <0.1 ml/min: AAV₂-hSyn-EYFP, AAV₂-hSyn-ChR2:EYFP, AAV₂-hSyn-NpHR3.0:EYFP, AAV₂-EF1a-DIO-Cre:EYFP, AAV₂-EF1a-DIO-ChR2: EYFP, or AAV₂-EF1a-DIO-NpHR3.0:EYFP (UNC vector core, USA). These viruses were injected into one of the following coordinates: the a cortical area near S1DZ (AP=−0.5 mm; ML=±2.5-3.0 mm; DV=0.9 mm), off-targets±0.5 mm or ±1.0 mm along the AP axis (AP=0.5 mm, ML=±3.0 mm, DV=0.9 mm; AP=0.0 mm, ML=±3.0 mm, DV=0.9 mm; AP=−1.0 mm, ML=±3.0 mm, DV=0.9 mm; and AP=−1.5 mm, ML=±3.0 mm, DV=0.9 mm), S1FL (AP=−0.5 mm, ML=±2.0 mm, DV=0.9 mm), or S1BF (AP=−0.5 mm, ML=±3.5 mm, DV=0.9 mm). Subsequently, fiber optic implants (300 μm core size, Thorlabs, USA) were bilaterally placed 400-500 mm above the virus injection sites.

For stimulating the specific S1DZ/TeA connection, rabies virus (RV-EYFP, RV-Chronos:EYFP or RV-ArchT:EYFP) was bilaterally introduced into the TeA (AP=−1.75 mm, ML=±4.15 mm, DV=1.5 mm) and optic fiber implants were placed onto the S1DZ (AP=−0.5 mm, ML=±2.5 mm, DV=0.3 mm). Fiber optic implants were then fixed in place with a small amount of dental cement (Lang Dental, USA) and the skin was glued back with Vetbond tissue adhesive (3M, USA).

For anterograde tracing, AAV₂-hSyn-EYFP was injected into the S1DZ and AAV₂-hSyn-mCherry into either the S1FL or S1BF.

Behavioral Analysis:

All behavioral training and testing were carried out in accordance with previously established behavioral schemes¹¹ with modifications. Experimenters were blind to the treatment group.

Ultrasonic Vocalizations:

On postnatal day 9, mouse pup ultrasonic vocalizations (USVs) were detected for 3 min using an Ultra-Sound Gate CM16/CMPA microphone (AviSoft, Germany) and SAS Prolab software (AviSoft, Germany) in a sound attenuation chamber under stable temperature (19-22° C.) and light control (15 lux).

Three-Chamber Social Approach Assay:

Male mice (8-12-weeks-old) were tested for sociability using a 3-chamber social approach paradigm. An empty object-containment cage (circular metallic cages, Stoelting Neuroscience) was each placed into the left and right chambers of a 3-chamber arena, which the experimental mice freely investigated for 10 min (exploration period). The following day, the mice underwent another 10 min exploration period. Immediately after, the mice were confined to the center chamber, while a social object (unfamiliar C57BL/6 male mouse) and an inanimate object (plastic toy) were placed alternatingly into either the left or right object-containment cage. Barriers to the adjacent chambers were removed, then the mice were allowed to explore the 3-chamber arena for 10 min. Approach behavior was defined as interaction time (i.e. sniffing, approach) with targets in each chamber (within 2 cm, excluding non-nose contact or exploration). Sessions were video-recorded and object approach behavior and total distance moved were analyzed using EthoVision tracking system (Noldus, Netherlands). Social preference index was calculated as the percentage of time spent investigating the social target out of the total approach behavior.

Marble Burying Test:

Mice were placed into testing arenas (arena size: 16″×8″×12″, bedding depth: 2″) each containing 20 glass marbles (laid out in four rows of five marbles equidistant from one another). At the end of the 15-min exploration period, mice were carefully removed from the testing cages and the number of marbles buried was recorded. The marble burying index was arbitrarily defined as the following: 1 for marbles covered>50% with bedding, 0.5 for ˜50% covered, or 0 for anything less.

Open Field Test:

Mice underwent a 15-min exploration period in the testing arena (arena size: 24″×24″×14). Sessions were video-recorded and analyzed for time spent in the center (center size: 12″×12″) using EthoVision Noldus tracking system (Noldus, Netherlands).

Behavioral Analysis with Optical Stimulation:

After two weeks of recovery, animals were assessed using the different behavioral schemes. During behavioral assays, animals were given 3 min of laser stimulation (On session, ChR2: 405 nm, 20 Hz, 50% duty cycle; Chronos: 488 nm, 20 Hz, 50% duty cycle; NpHR3.0 and ArchT: 595 nm, 20 Hz, 50% duty cycle) followed by 3 min of no stimulation (Off session). Animals started with either an On or Off session in a counterbalanced manner. Photostimulation was controlled with a waveform generator (Keysight, 33220A, USA) and Ethovision XT (Noldus, Netherlands). Behavioral analysis was conducted as described earlier using EthoVision Noldus tracking system (Noldus, Netherlands).

For quantitative analysis of photostimulation-dependent activation of virus expressing neurons, mice were sacrificed 1 hr after the end of the behavioral testing. Brain slices were double-labeled for c-Fos (sc-7270, Santa Cruz, USA) and EYFP (ab5450, Abcam, USA). The percentage of neurons expressing c-Fos (c-Fos+EYFP⁺/EYFP⁺) within a 500 μm×500 μm area, 300 μm below the optical fiber placement was calculated. Results from mice without viral infection or with inaccurate targeting of virus or fiber implantations were excluded.

Slice Preparation for Whole-Cell Electrophysiology:

Mice were anesthetized with pentobarbital (40 mg/kg i.p.) and intracardially perfused with ice-cold dissection buffer (in mM: 87 NaCl, 2.5 KCl, 1.25 NaH₂PO₄, 26 NaHCO₃, 75 sucrose, 10 dextrose, 1.3 ascorbic acid, 7 MgCl₂ and 0.5 CaCl₂) bubbled with 95% O₂-5% CO₂. Brains were rapidly removed and immersed in ice-cold dissection buffer. Somatosensory cortical sections were dissected and 300 μm coronal slices were prepared using a Leica VT1200S vibratome (Leica, USA). Slices recovered for 20 min in a 35° C. submersion chamber filled with oxygenated artificial cerebrospinal fluid (aCSF) (in mM: 124 NaCl, 3 KCl, 1.25 NaH₂PO₄, 26 NaHCO₃, 1 MgCl₂, 2 CaCl₂, and 20 glucose) and then kept at room temperature for >40 min until use.

Voltage-Clamp Recordings

Miniature Inhibitory Postsynaptic Currents:

To specifically isolate miniature inhibitory postsynaptic currents (mIPSCs), slices were placed in a submersion chamber maintained at 32° C., perfused at 2 ml/min with oxygenated aCSF (as described above) containing 1 μM TTX, 100 μM DL-APV, and 20 μM DNQX and held at 0 mV. Cells were visualized using an Olympus BX-51 equipped with infrared differential interference contrast (IR-DIC) optics. Pyramidal neurons were identified by intrinsic membrane properties present in layer II/III of S1DZ and morphological confirmation of spiny dendrites. Patch pipettes were pulled from thick-walled borosilicate glass (P-2000, Sutter Instruments Novato, USA). Open tip resistances were between 2.5-6MΩ and were back-filled with an internal containing the following (in mM): 100 CsCH₃SO₃, 15 CsCl, 2.5 MgCl₂, 10 Hepes, 5 QX-314, 5 BAPTA, 4 Mg-ATP, 0.3 Mg-GTP, and 0.025 Alexa-568 with pH adjusted to 7.25 with 1M CsOH and osmolarity adjusted to ˜295 mOsm by the addition of sucrose. Voltage-clamp recordings were performed in whole-cell configuration using patch-clamp amplifier (Multiclamp 700B, Molecular Devices) and data were acquired and analyzed using pClamp 10 software (Molecular Devices). Pipette seal resistances were >1GΩ and pipette capacitive transients were minimized before breakthrough. Changes in series and input resistance were monitored throughout the experiment by giving a test pulse every 30 s and measuring the amplitude of the capacitive current. A maximum of one cell was recorded per slice, which was subsequently fixed in 4% PFA for post-hoc validation of cortical patch location. Cells were discarded if series resistance rose above 20MΩ and/or if post-hoc validation revealed cells recorded from were outside of the cortical patch. The experimenter was blinded during the acquisition and analysis of the postsynaptic currents.

Immunohistochemistry

For cryosectioning, animals were intracardially perfused, and the brain was dissected out, fixed with 4% paraformaldehyde in PBS overnight at 4° C., and cryoprotected in 30% sucrose solution. The left hemisphere was marked with a needle and the sections were coronally sliced at 40 μm using a cryostat (Leica, USA). For vibratome sectioning, animals were intracardially perfused, and the brain was fixed with 4% paraformaldehyde in PBS overnight at 4° C. The brains were coronally sliced at 50 μm with a Leica VT1000S vibratome (Leica, USA).

Slices were permeabilized with blocking solution containing 0.4% Triton X-100 and 2% goat serum in PBS for 1 h at room temperature (RT) and then incubated with anti-rabbit-TBR1 (ab31940, Abcam, USA), anti-mouse-SATB2 (ab51502, Abcam, USA), anti-rabbit-PV (PV27, Swant, Switzerland), anti-rabbit-VIP (20077, Immunostar, USA), anti-mouse-NeuN (MAB377X, Millipore, USA), or anti-rabbit-c-Fos (sc-7270, Santa Cruz, USA) antibodies overnight at 4° C. The following day, slices were incubated with fluorescently conjugated secondary antibodies (Invitrogen, USA) for 1 hr at RT with Neurotrace (Invitrogen, USA), and mounted in Vectashield mounting medium containing 4′,6-Diamidino-2-Phenylindole, Dihydrochloride (DAPI; Vector laboratories, USA). Images of stained slices were acquired using a confocal microscope (LSM710; Carl Zeiss, Germany) with a 20× objective lens; all image settings were kept constant across experimental groups.

For anterograde-tracing, brains were sliced into 100 μm sections, and the prepared slices were labeled with anti-chicken-GFP (ab5450, Abcam), anti-rabbit-DsRed (632496, Clontech) and DAPI. Images were acquired with a confocal microscope and then aligned to the Paxinos brain atlas.

Analysis of the Cortical Patches

Cortical patches were identified by the absence of SATB2 or TBR1 expression. Spatial locations of the cortical patches were determined based on their distance from the midline of the brain and the layer structures of the cortex. These locations were matched to their corresponding regions in a mouse brain atlas (Paxinos brain atlas). The size of the cortical patches was calculated using Zen software (Carl Zeiss, Germany).

Cell types within the cortical patches of the 51 were characterized by staining brain slices from PBS and MIA offspring for SATB2 or TBR1, PV, VIP and NeuN. The cortical region of interest, centered on a cortical patch in MIA offspring or the corresponding area in PBS offspring, was divided into 10 equal laminar blocks (bin) representing different depths of the cortex. Individual marker positive cells (SATB2, PV, VIP, or NeuN) were quantified manually. Experimenter was blind to the treatment groups.

Statistics

Statistical analyses were performed using Prism software. One-way and Two-way ANOVAs were followed by Tukey or Sidak corrections. All data are represented as mean±s.e.m. Criteria for sample siz and exclusion of animal data were pre-established¹¹. When conducting behavioral assays, treatment cages were pseudo-randomly assigned for testing order.

Results

As described herein, the present inventors determined that cortical patches in the brains of adult MIA offspring were found predominantly in the primary somatosensory cortex (S1), as well as in the secondary motor cortex (M2) and other cortical regions, including the temporal association area (TeA). See, for example, FIGS. 1A, 5, and 6. Further analysis revealed that the cortical patches most consistently centered on S1DZ, a region of the primary somatosensory cortex that is morphologically characterized by the absence of a discernible 4th cortical layer and functionally to muscle- and joint-related responses. See, for example, FIG. 1C and FIG. 7.

The present inventors also found that S1 cortical patches of MIA offspring display a specific loss of PV⁺ cortical neurons, but observed no significant differences in the expression of the neuronal specific marker NeuN or of the vasoactive intestinal polypeptide (VIP), which is expressed in interneurons derived from the caudal ganglionic eminence, between PBS control and MIA offspring. See, for example, FIG. 1D, E. To test whether this selective loss of PV⁺ interneurons results in a diminished inhibitory drive onto S1 pyramidal neurons, whole-cell patch clamp recordings were used to measure the mIPSCs in S1 layer II/III pyramidal neurons of PBS or MIA offspring. These investigations revealed a reduction in frequency, but not amplitude, of mIPSC paralleled by an increase in the number of S1 neurons expressing c-Fos, a marker of neuronal activation. See, for example, FIGS. 1F,G and 8.

The present inventors, furthermore, identified a developmental window in which time or by which time MIA can be induced and result in the development of MIA-associated behaviors and the appearance of cortical patches. In brief, injection of poly(I:C) into pregnant dams at embryonic stages E12.5, E15.5, or E18.5 and assessment of offspring generated thereafter for MIA-associated behavioral phenotypes revealed that pups from pregnant dams injected with poly(I:C) at E12.5 (MIA offspring) emitted more ultrasonic vocalization (USV) calls than pups from PBS-injected dams (PBS offspring). This finding was not observed in the offspring from dams injected with poly(I:C) either at E15.5 or E18.5 (FIG. 9D). In similar fashion, repetitive behaviors using the marble-burying assay, natural inclination towards social targets with the sociability assay as well as anxiety-related behaviors by measuring the time spent by adult MIA offspring at the center of an open field were assessed and deficits were observed in the offspring when exposed to prenatal MIA at E12.5, but not when exposed at either E15.5 or E18.5. See, for example, FIG. 9. These results suggest that all behavioral abnormalities emerge from a discrete developmental stage.

It is also noteworthy that the size of the S1 cortical patches correlated with the severity of behavioral phenotypes. More particularly, the size of S1 cortical patches positively correlated with the marble burying index, while negatively correlated with both sociability and time spent in the center of an open field. See, for example, FIG. 2. Knocking-out IL-17Ra in offspring using a Cre driver line specific for the nervous system—Nestin-Cre—prevented the development of S1 cortical patches. See, for example, FIG. 10. Together, these data collectively reveal that the presence of cortical patches is highly predictive of MIA-induced behaviors and further suggest that timing of inflammation, as well as IL-17Ra expression in the fetal brain dictate the severity of ASD-like behavioral phenotypes in offspring by contributing to the formation of 51 cortical patches.

The characterization of cortical patches indicated that an increase in the overall neural activity within S1 could be a major factor driving abnormal behavioral phenotypes in MIA offspring. See, for example, FIGS. 1f, g, and 8. Results presented herein demonstrate that increasing neural activity in S1 recapitulates MIA-induced behaviors in WT adult animals. See, for example, FIGS. 3 and 11A. More particularly, increasing activity specifically in excitatory glutamatergic neurons was shown to recapitulate all three MIA-associated behaviors. See, for example, FIGS. 3 and 12. Inhibiting the activity of PV⁺ neurons, moreover, mimicked the loss of PV⁺ neurons observed in the MIA-cortical patches and recapitulated all three MIA-associated behavioral phenotypes. See, for example, FIGS. 3 and 13.

Additional results presented herein demonstrate that MIA-like behaviors can be recapitulated in WT animals either through the activation of excitatory neurons or the inhibition of PV⁺ inhibitory neurons and that this feature is primarily localized to the cortical region centered on the S1DZ. See, for example, FIGS. 14 and 15.

Results presented herein also demonstrate that acute reduction in neural activity in the S1DZ region is sufficient to rescue ASD-like behavioral phenotypes in MIA offspring prenatally exposed to maternal inflammation, See, for example, FIGS. 4 and 16.

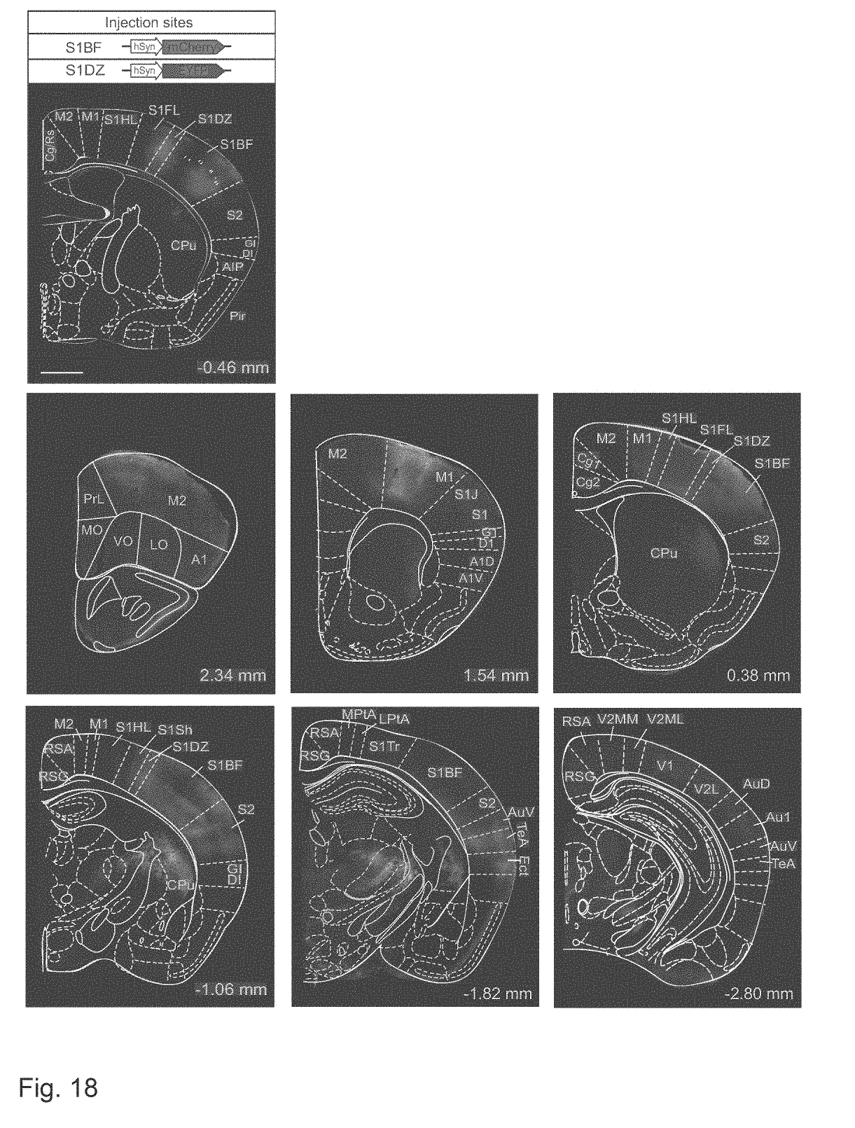

An investigation into the efferent targets of the S1DZ revealed that the neural connection from the S1DZ to the TeA selectively modulates interactions with social targets without impacting repetitive behaviors. See, for example, FIGS. 4 and 17-19.

The findings presented herein, therefore, identify specific regions of the brain (e.g., S1DZ) wherein E/I dysregulation contributes causally to ASD and ASD-like symptoms and, moreover, points to modes of therapeutic intervention that can be implemented to confer benefit to subjects in need thereof.

This invention may be embodied in other forms or carried out in other ways without departing from the spirit or essential characteristics thereof. The present disclosure is therefore to be considered as in all aspects illustrative and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

### REFERENCES

1 Atladóttir, H. O. et al. Maternal infection requiring hospitalization during pregnancy and autism spectrum disorders. J Autism Dev Disord. 40, 1423-1430 (2010).
2 Patterson, P. H. Immune involvement in schizophrenia and autism:etiology, pathology and animal models. Behav Brain Res 7, 313-321 (2009).
3 Brown, A. S. et al. Elevated maternal C-reactive protein and autism in a national birth cohort. Mol Psychiatry 19, 259-264 (2014).
4 Atladóttir, H. O. et al. Association of family history of autoimmune diseases and autism spectrum disorders. Pediatrics 124, 687-694 (2009).
5 Ashwood, P., Wills, S. & Van de Water, J. The immune response in autism: a new frontier for autism research. J Lekoc Biol 80, 1-15 (2006).
6 Lee, B. K. et al. Maternal hospitalization with infection during pregnancy and risk of autism spectrum disorders. Brain Behav Immun 44, 199-105 (2015).
7 Smith, S. E., Li, J., Garbett, K., Mimics, K. & Patterson, P. H. Maternal immune activation alters fetal brain development through interleukin-6. J Neurosci 27, 10695-10702 (2007).
8 Meyer, U. & Feldon, J. Neural basis of psychosis-related behaviour in the infection model of schizophrenia. Behav Brain Res 204, 322-334 (2009).
9 Shi, L., Fatemi, S. H., Sidwell, R. W. & Patterson, P. H. Maternal influenza infection causes marked behavioral and pharmacological changes in the offspring. J Neurosci 23, 297-302 (2003).
10 Malkova, N. V., Yu, C. Z., Hsiao, E. Y., Moore, M. J. & Patterson, P. H. Maternal immune activation yields offspring displaying mouse versions of the three core symptoms of autism. Brain Behav Immun 26, 607-616 (2012).
11 Choi, G. B. et al. The maternal interleukin-17a pathway in mice promotes autism-like phenotypes in offspring. Science 351, 933-939, doi:10.1126/science.aad0314 (2016).
12 Stoner, R. et al. Patches of disorganization in the neocortex of children with autism. N Engl J Med 27, 1209-1219 (2014).
13 Paxinos, G. & Franklin, K. The mouse brain in stereotaxic coordinates. (2004).
14 Lee, T. & Kim, U. Descending projections from the dysgranular zone of rat primary somatosensory cortex processing deep somatic input. J Comp Neurol 520, 1021-1046 (2012).
15 Welker, W., Sanderson, K. J. & Shambes, G. M. Patterns of afferent projections to transitional zones in the somatic sensorimotor cerebral cortex of albino rats. Brain Res 292, 261-267 (1984).
16 Chapin, J. K. & Lin, C. S. Mapping the body representation in the S1 cortex of anesthetized and awake rats. J Comp Neurol 229, 199-213 (1984).
17 Gogolla, N. et al. Common circuit defect of excitatory-inhibitory balance in mouse models of autism. J Neurodev Disord 1, 172-181 (2009).
18 Orefice, L. L. et al. Peripheral Mechanosensory Neuron Dysfunction Underlies Tactile and Behavioral Deficits in Mouse Models of ASDs. Cell 166, 299-313 (2016).
19 Selby, L., Zhang, C. & Sun, Q. Q. Major defects in neocortical GABAergic inhibitory circuits in mice lacking the fragile X mental retardation protein. Neurosci Lett 412, 227-232 (2007).
20 Kepecs, A. & Fishell, G. Interneuron cell types are fit to function. Nature 605, 318-326 (2014).
21 Mullen, R. J., Buck, C. R. & Smith, A. M. NeuN, a neuronal specific nuclear protein in vertebrates. Development 116, 201-211 (1992).
22 Rubenstein, J. L. & Merzenich, M. M. Model of autism: increased ratio of excitation/inhibition in key neural systems. Genes Brain Behav 2, 255-267 (2003).
23 Chao, H. T. et al. Dysfunction in GABA signalling mediates autism-like stereotypies and Rett syndrome phenotypes. Nature 468 (2010).
24 Han, S., Tai, C., Jones, C. J., Scheuer, T. & Catterall, W. A. Enhancement of inhibitory neurotransmission by GABAA receptors having α2,3-subunits ameliorates behavioral deficits in a mouse model of autism. Neuron 81, 1282-1289 (2014).
25 Peñagarikano, O. et al. Absence of CNTNAP2 leads to epilepsy, neuronal migration abnormalities, and core autism-related deficits. Cell 147, 235-246 (2011).
26 Nelson, S. B. & Vallakh, V. Excitatory/Inhibitory Balance and Circuit Homeostasis in Autism Spectrum Disorders. Neuron 87, 684-698 (2015).
27 Wohr, M. et al. Lack of parvalbumin in mice leads to behavioral deficits relevant to all human autism core symptoms and related neural morphofunctional abnormalities. Transl Psychiatry 10, e525 (2015).
28 Canetta, S. et al. Maternal immune activation leads to selective functional deficits in offspring parvalbumin interneurons. Mol Psychiatry 21, 956-968 (2016).
29 Zhang, F., Wang, L. P., Boyden, E. S. & Deisseroth, K. Chrannelrhodopsin-2 and optical control of excitable cells. Nat Methods 3, 785-792 (2006).
30 Zhang, F. et al. Multimodal fast optical interrogation of neural circuitry. Nature 446, 633-639 (2007).
31 Vong, L. et al. Leptin action on GABAergic neurons prevents obesity and reduces inhibitory tone to POMC neurons. Neuron 71, 142-154 (2011).
32 Hippenmeyer, S. et al. A developmental switch in the response of DRG neurons to ETS transcription factor signaling. PLoS Biol 3, e159 (2005).
33 Wickersham, I. R., Finke, S., Conzelmann, K. & Callaway, E. M. Retrograde neuronal tracing with a deletion-mutant rabies virus. Nat Methods 4, 47-49 (2007).
34 Klapoetke, N. C. et al. Independent optical excitation of distinct neural populations. Nat Methods 11, 338-346 (2014).
35 Chow, B. Y. et al. High-performance genetically targetable optical neural silencing by light-driven proton pumps. Nature 463, 98-102 (2010).
36 Cascio, C. J., Foss-Feig, J. H., Burnette, C. P., Heacock, J. L. & Cosby, A. A. The rubber hand illusion in children with autism spectrum disorders: delayed influence of combined tactile and visual input on proprioception. Autism 16, 406-419 (2012).
37 Haswell, C. C., Izawa, J., Dowell, L. R., Mostofsky, S. H. & Shadmehr, R. Representation of internal models of action in the autistic brain. Nat Neurosci 12, 970-972 (2009).
38 Minshew, N. J., Sung, K., Jones, B. L. & Furman, J. M. Underdevelopment of the postural control system in autism. Neurology 63, 2056-2061 (2004).
39 Glazebrook, C., Gonzalez, D., Hansen, S. & Elliott, D. The role of vision for online control of manual aiming movements in persons with autism spectrum disorders. Autism 13, 411-433 (2009).
40 El Malki, K. et al. An alternative pathway of imiquimod-induced psoriasis-like skin inflammation in the absence of interleukin-17 receptor a signaling. J Invest Dermatol 133, 441-451 (2013).

1. A method for treating a psychiatric disorder in a subject, the method comprising the steps of selecting a subject afflicted with or at risk for developing the psychiatric disorder based on in utero exposure to maternal immune activation (MIA) and treating the subject by administering a pharmacological agent or implementing optogenetic or chemogenetic tools that corrects dysregulated neuronal excitation/inhibition (E/I) ratios in the cortex of the subject.

2. A method for treating a psychiatric disorder in a subject, the method comprising the steps of selecting a subject afflicted with or at risk for developing the psychiatric disorder based on the presence of patches exhibiting dysregulated neuronal excitation/inhibition (E/I) ratios in the cortex of the subject and treating the subject by administering a pharmacological agent or implementing optogenetic or chemogenetic tools that corrects the dysregulated neuronal E/I ratios in the cortex of the subject.

3. The method of claim 1, wherein the psychiatric disorder is autism spectrum disorder, schizophrenia, or depression.

4. The method of claim 3, wherein the autism spectrum disorder is autism.

5. (canceled)

6. The method of claim 1, wherein the MIA is associated with a hyper-inflammatory condition.

7. The method of claim 6, wherein the hyper-inflammatory condition is associated with a viral or bacterial infection or exposure to an inflammatory or environmental toxin during pregnancy.

8. The method of claim 1, wherein the in utero exposure to MIA occurs in the first trimester, second trimester, or third trimester of a pregnancy in a mammal.

9. The method of claim 1, wherein the in utero exposure to MIA occurs in the late first trimester or the second trimester of a pregnancy in a mammal.

10. The method of claim 1, wherein the mammal is a human.

11. The method of claim 1, further comprising evaluating the cortex of the subject for the presence of patches exhibiting dysregulated neuronal excitation/inhibition (E/I) ratios.

12. The method of claim 2, wherein at least some of the patches exhibiting dysregulated neuronal excitation/inhibition (E/I) ratios are in the primary somatosensory cortex of the subject.

13. The method of claim 1, wherein the pharmacological agent is a GABAergic receptor agonist.

14. The method of claim 13, wherein the GABAergic receptor agonist is synthetic GABA, muscimol, a barbiturate, or a benzodiazapine.

15. The method of claim 1, wherein the optogenetic tools are channelrhodopsin, halorhodopsin, or an OptoXR.

16. The method of claim 1, wherein the pharmacological agent is a modulator of interleukin-17 (IL-17) activity or a modulator of interferon-gamma (IFN-γ) activity.

17-21. (canceled)

22. The method of claim 1, wherein the pharmacological agent or the optogenetic or chemogenetic tools are administered or delivered to the cortex of the subject.

23. The method of claim 1, wherein the pharmacological agent or the optogenetic or chemogenetic tools are administered or delivered to the primary somatosensory cortex of the subject.

24-31. (canceled)

32. A method for treating a psychiatric disorder in a subject, the method comprising the steps of selecting a subject afflicted with the psychiatric disorder and treating the subject by administering a modulator of IL-17 activity and/or a modulator of IFN-γ activity that corrects dysregulated neuronal excitation/inhibition (E/I) ratios in the cortex of the subject.

33. The method of claim 32, wherein the subject is selected for the presence of patches of dysregulated neuronal excitation/inhibition (E/I) ratios in the primary somatosensory cortex.

34-39. (canceled)

40. The method of claim 32, wherein the subject is selected based on in utero exposure to MIA.

41-43. (canceled)
